(19) United States
(12) Patent Application Publication
(10) Pub. No.: US 2022/0119517 A1
(43) Pub. Date: Apr. 21, 2022

(54) ANTI-CLAUDIN18.2 ANTIBODY AND USE THEREOF

(71) Applicant: NANJING SANHOME PHARMACEUTICAL CO., LTD., Nanjing (CN)

(72) Inventors: Yong Wang, Nanjing (CN); Liwen Zhao, Nanjing (CN); Zhenzhen Lu, Nanjing (CN); Shichao Sun, Nanjing (CN); Yuhong Pang, Nanjing (CN); Jian Wei, Nanjing (CN); Qiuhua Zhou, Nanjing (CN); Luwei Han, Nanjing (CN); Qifeng Song, Nanjing (CN)

(21) Appl. No.: 17/271,033

(22) PCT Filed: Aug. 26, 2019
(86) PCT No.: PCT/CN2019/102502

§ 371 Date: Feb. 24, 2021

(30) Foreign Application Priority Data
Aug. 27, 2018 (CN) 201810984724.2

(51) Int. Cl. C07K 16/28; A61P 35/00

(52) U.S. Cl. CPC C07K 16/28; A61P 35/00; C07K 2317/52; C07K 2317/92; C07K 2317/732; C07K 2317/24

(57) ABSTRACT

Provided are an anti-Claudin18.2 antibody or an antigen binding fragment thereof, a pharmaceutical composition comprising an anti-Claudin18.2 antibody or an antigen binding fragment thereof, and uses thereof. The antibody and Claudin18.2 have significant binding activity and affinity, have efficient in-vitro ADCC cytotoxic activity against a tumor cell, and can be applied in the preparation of an antineoplastic drug.

### FIELD OF THE INVENTION

The present application relates to the technical field of antibody drugs, in particular to an anti-Claudin18.2 antibody or an antigen binding fragment thereof, a pharmaceutical composition comprising an anti-Claudin18.2 antibody or an antigen binding fragment thereof, and uses thereof.

### BACKGROUND OF THE INVENTION

Gastric cancer is a type of cancer with the fifth highest incidence rate in the world, and its fatality rate is the third highest. Currently, chemotherapy is the standard first-line treatment for advanced or recurrent gastric cancer. More than 90% cancer patients will receive ordinary chemotherapy and targeted chemotherapy, however, chemotherapy generally cannot cure cancer, it only can prolong the survival time of patients and improve their quality of life, and it is easy to develop drug resistance. Biological targeted therapy usually targets tumor-specific antigens or tumor-related antigens, and relies on the binding or blocking of antibodies to the target to trigger the immune response in the body, selectively killing the tumor without affecting the surrounding normal cells and tissues. Targeted therapy has strong specificity, few side effects and precise efficacy.

However, due to the heterogeneity of malignant tumors, different cancers show different molecular biology characteristics. Especially for gastric cancer, of which the degree of heterogeneity is relatively high, the biological targeted therapeutic drugs developed in the past for gastric cancer when used alone are basically ineffective or effective for a very small portion of patients. The reason is that those targets targeted by these monoclonal antibody drugs are only expressed in specific subtypes of gastric cancer patients. A typical example is Trastuzumab, which has a good efficacy when combined with chemotherapy, but only can apply to HER2-positive individuals, which only account for 15% of all gastric cancer patients.

Claudins are the most important skeleton proteins that determine the tight junction structure between cells, which are involved in adherent junction and play an important role in the metastasis and invasion of tumor cells. Claudin proteins are widely distributed in mammalian epithelial and endothelial cells, mainly on the side of epithelial cells and on the plasma membrane of basal cells.

Different Claudin proteins have their own specific expression in different tissues, wherein the Claudin18 (CLDN18) gene is located at 3q22.3, has a molecular weight of 24 kDa, contains 261 amino acid residues, is a member of the Claudin superfamily and its protein structure contains 2 extracellular loops and 4 transmembrane domains. The two subtypes of human CLDN18 protein are CLDN18.1 (NM_016369.3) and CLDN18.2 (NM_001002026.2), respectively, and in the primary structure sequence of both proteins, there are only differences in the amino acid residues in certain positions of the structure of the N-terminal signal peptide to the extracellular loop 1 (Loop1), and in particular in the extracellular loop 1, CLDN18.1 and CLDN18.2 differ only by 8 amino acids. In addition, the sequence homology among different species of the two subtype proteins of CLDN18 is also very high. Among them, the extracellular loop 1 of CLDN18.2 has the exactly same sequence in different species such as human, mouse, and macaque, while the CLDN18.2 protein homology between human and mouse reaches 84%, indicating that the CLDN18.2 protein sequence is extremely conservative (O. Tureci., et al., Gene 481: 83-92, 2011).

The two subtype proteins of CLDN18 are expressed and exert their physiological functions in different tissues, respectively. CLDN18.1 is constitutively expressed in normal lung tissue cells; whereas CLDN18.2 protein is expressed in normal gastric epithelial cell membranes, and is not expressed or expressed in very low levels in various tissues except the stomach. CLDN 18.2 was detected positive by Salin., et al in a variety of cancer tissues including gastric cancer, pancreatic cancer, and esophageal cancer using RT-PCR and immunohistochemical methods, with the overall positive rate reaching 62.7% (U. Salin., et al., Clin Cancer Res 14(23): 7624-34, 2008). Other studies have shown that CLDN18.2 protein expressed by normal gastric parietal cells has homogenous glycosylation modification and compact structures between the cells. However, the structures between gastric cancer cells are loose, and the cells are easy to invade and metastasize, and the CLDN18.2 protein expressed thereon is incompletely glycosylated, which may lead to target exposure (WO2004/047863). Therefore, CLDN18.2 may be an ideal tumor-associated antigen target.

Currently, Astellas Pharma Inc. has developed a monoclonal antibody drug Zolbetuximab (also known as IMAB362) targeting human Claudin18.2, which has significant clinical efficacy. Phase II clinical trials have shown that (S. Batran. et al., Journal of Clinical Oncology 34 (no.18_suppl), 2016), in the treatment of advanced or recurrent gastric cancer using the combination of monoclonal antibody drugs and chemotherapeutic drugs, the median survival time of patients was 13.2 months, significantly longer than the 8.4 months using chemotherapy alone. It is preliminarily confirmed that specific antibodies targeting CLDN18.2 can significantly improve the progression-free survival time and overall survival time of patients with advanced gastric cancer, adenocarcinoma of gastroesophageal junction.

The preparation of a human-mouse chimeric monoclonal antibody described in WO2004/047863 is the first therapeutic antibody developed against the human Claudin18.2 target (hereinafter referred to as anti-CLDN18.2 antibody) worldwide. The antibody described in that patent application specifically kills CLDN18.2 positive tumor cells mainly through the antibody-dependent cell-mediated cytotoxicity (ADCC) pathway. However, the specific recognition and killing between the antibody and tumor cells described in that patent application mainly depends on the abundance of the CLDN18.2 target expressed on the tumor cells, and relatively obvious binding and cytotoxic activity are only shown when the expression of CLDN18.2 on the cell surface is high. This indicates that it is necessary to prepare a high-affinity monoclonal antibody to improve the ability to selectively kill tumor cells.

Based on the prior art above, the present invention now provides an efficient CLDN18.2 antibody or an antigen binding fragment thereof, which has excellent anti-tumor activity.

### SUMMARY OF THE INVENTION

One aspect of the present invention provides an anti-CLDN18.2 antibody or an antigen binding fragment thereof, which comprises a heavy chain variable region and/or a light chain variable region, wherein the heavy chain variable region comprises complementarity determining region 1 of the heavy chain variable region (HCDR1), complementarity determining region 2 of the heavy chain variable region (HCDR2), and/or complementarity determining region 3 of the heavy chain variable region (HCDR3), and the light chain variable region comprises complementarity determining region 1 of the light chain variable region (LCDR1), complementarity determining region 2 of the light chain variable region (LCDR2) and/or complementarity determining region 3 of the light chain variable region (LCDR3).

In some embodiments, the present invention provides an anti-CLDN18.2 antibody or an antigen binding fragment thereof, which comprises a heavy chain variable region and a light chain variable region, wherein:

(1) the heavy chain variable region comprises HCDR1, HCDR2 and HCDR3:

(a1) the amino acid sequence of the HCDR1 is selected from SEQ ID NO 101, SEQ ID NO 102, SEQ ID NO 103 and amino acid sequences having at least 85% sequence identity with them;

(a2) the amino acid sequence of the HCDR2 is selected from SEQ ID NO 104, SEQ ID NO 105 and amino acid sequences having at least 85% sequence identity with them;

(a3) the amino acid sequence of the HCDR3 is selected from SEQ ID NO 106, SEQ ID NO 107 and amino acid sequences having at least 85% sequence identity with them; and

(2) the light chain variable region comprises LCDR1, LCDR2 and LCDR3, wherein:

(a4) the amino acid sequence of the LCDR1 is SEQ ID NO 108;

(a5) the amino acid sequence of LCDR2 is selected from SEQ ID NO 109, SEQ ID NO 110, SEQ ID NO 111 and amino acid sequences having at least 85% sequence identity with them;

(a6) the amino acid sequence of the LCDR3 is selected from SEQ ID NO 112, SEQ ID NO 113, SEQ ID NO 114, SEQ ID NO 115, SEQ ID NO 116 and amino acid sequences having at least 85% sequence identity with them.

In one specific embodiment, the present invention provides an anti-CLDN18.2 antibody or an antigen binding fragment thereof, which has a heavy chain variable region with the HCDR1, HCDR2, and HCDR3 being SEQ ID NO 101, 105 and 106, respectively, or CDRs having at least 85% sequence identity with amino acid sequences shown in SEQ ID NO 101, 105 and 106, and a light chain variable region with the LCDR1, LCDR2, and LCDR3 being SEQ ID NO 108, 111 and 115, respectively, or CDRs having at least 85% sequence identity with amino acid sequences shown in SEQ ID NO 108, 111 and 115.

In some specific embodiments, the anti-CLDN18.2 antibody or the antigen binding fragment thereof according to the present invention is a monoclonal antibody or an antigen binding fragment thereof.

In some specific embodiments, the anti-CLDN18.2 antibody or the antigen binding fragment thereof according to the present invention is a murine-derived antibody or an antigen binding fragment thereof, a chimeric antibody or an antigen binding fragment thereof, or a humanized antibody or an antigen binding fragment thereof.

In some specific embodiments, the present invention provides an anti-CLDN18.2 antibody or an antigen binding fragment thereof, which comprises a heavy chain variable region and a light chain variable region, wherein:

(1) the amino acid sequence of the heavy chain variable region is selected from:

(b1) an amino acid sequence shown in SEQ ID NO 50, SEQ ID NO 72, SEQ ID NO 74, SEQ ID NO 77, SEQ ID NO 79;

(b2) an amino acid sequence obtained by substituting, deleting or adding one or more amino acids of the amino acid sequences shown in (b1), and with the same or similar function as the amino acid sequences shown in (b1); and

(b3) an amino acid sequence having at least 80% sequence identity with the amino acid sequences shown in (b1); and

(2) the amino acid sequence of the light chain variable region is selected from:

(b4) an amino acid sequence shown in SEQ ID NO 51, SEQ ID NO 70, SEQ ID NO 71, SEQ ID NO 73, SEQ ID NO 75, SEQ ID NO 76, SEQ ID NO 78, SEQ ID NO 80, SEQ ID NO 81;

(b5) an amino acid sequence obtained by substituting, deleting or adding one or more amino acids of the amino acid sequences shown in (b4), and with the same or similar function as the amino acid sequences shown in (b4); and

(b6) an amino acid sequence having at least 80% sequence identity with the amino acid sequences shown in (b4).

In some specific embodiments, the present invention provides an anti-CLDN18.2 antibody or an antigen binding fragment thereof, wherein the amino acid sequence of the heavy chain variable region is SEQ ID NO:50, the amino acid sequence obtained by substituting, deleting or adding one or more amino acids of SEQ ID NO:50 and with the same function as SEQ ID NO:50 or the amino acid sequence having at least 85% sequence identity with SEQ ID NO:50, and the amino acid sequence of the light chain variable region is SEQ ID NO:51, the amino acid sequence obtained by substituting, deleting or adding one or more amino acids of SEQ ID NO:51 and with the same function as SEQ ID NO:51 or the amino acid sequence having at least 85% sequence identity with SEQ ID NO:51.

In some specific embodiments, the present invention provides an anti-CLDN18.2 antibody or an antigen binding fragment thereof, wherein the amino acid sequence of the heavy chain variable region is SEQ ID NO:50, the amino acid sequence obtained by substituting, deleting or adding one or more amino acids of SEQ ID NO:50 and with the same function as SEQ ID NO:50 or the amino acid sequence having at least 85% sequence identity with SEQ ID NO:50, and the amino acid sequence of the light chain variable region is SEQ ID NO:70, the amino acid sequence obtained by substituting, deleting or adding one or more amino acids of SEQ ID NO:70 and with the same function as SEQ ID NO:70 or the amino acid sequence having at least 85% sequence identity with SEQ ID NO:70.

In some specific embodiments, the present invention provides an anti-CLDN18.2 antibody or an antigen binding fragment thereof, wherein the amino acid sequence of the heavy chain variable region is SEQ ID NO:50, the amino acid sequence obtained by substituting, deleting or adding one or more amino acids of SEQ ID NO:50 and with the same function as SEQ ID NO:50 or the amino acid sequence having at least 85% sequence identity with SEQ ID NO:50, and the amino acid sequence of the light chain variable region is SEQ ID NO:71, the amino acid sequence obtained by substituting, deleting or adding one or more amino acids of SEQ ID NO:71 and with the same function as SEQ ID NO:71 or the amino acid sequence having at least 85% sequence identity with SEQ ID NO:71.

In some specific embodiments, the present invention provides an anti-CLDN18.2 antibody or an antigen binding fragment thereof, wherein the amino acid sequence of the heavy chain variable region is SEQ ID NO:72, the amino acid sequence obtained by substituting, deleting or adding one or more amino acids of SEQ ID NO: 72 and with the same function as SEQ ID NO:72 or the amino acid sequence having at least 85% sequence identity with SEQ ID NO:72, and the amino acid sequence of the light chain variable region is SEQ ID NO:73, the amino acid sequence obtained by substituting, deleting or adding one or more amino acids of SEQ ID NO:73 and with the same function as SEQ ID NO:73 or the amino acid sequence having at least 85% sequence identity with SEQ ID NO:73.

In some specific embodiments, the present invention provides an anti-CLDN18.2 antibody or an antigen binding fragment thereof, wherein the amino acid sequence of the heavy chain variable region is SEQ ID NO:74, the amino acid sequence obtained by substituting, deleting or adding one or more amino acids of SEQ ID NO: 74 and with the same function as SEQ ID NO:74 or the amino acid sequence having at least 85% sequence identity with SEQ ID NO:74, and the amino acid sequence of the light chain variable region is SEQ ID NO:75, the amino acid sequence obtained by substituting, deleting or adding one or more amino acids of SEQ ID NO:75 and with the same function as SEQ ID NO:75 or the amino acid sequence having at least 85% sequence identity with SEQ ID NO:75.

In some specific embodiments, the present invention provides an anti-CLDN18.2 antibody or an antigen binding fragment thereof, wherein the amino acid sequence of the heavy chain variable region is SEQ ID NO:50, the amino acid sequence obtained by substituting, deleting or adding one or more amino acids of SEQ ID NO:50 and with the same function as SEQ ID NO:50 or the amino acid sequence having at least 85% sequence identity with SEQ ID NO:50, and the amino acid sequence of the light chain variable region is SEQ ID NO:76, the amino acid sequence obtained by substituting, deleting or adding one or more amino acids of SEQ ID NO:76 and with the same function as SEQ ID NO:76 or the amino acid sequence having at least 85% sequence identity with SEQ ID NO:76.

In some specific embodiments, the present invention provides an anti-CLDN18.2 antibody or an antigen binding fragment thereof, wherein the amino acid sequence of the heavy chain variable region is SEQ ID NO:77, the amino acid sequence obtained by substituting, deleting or adding one or more amino acids of SEQ ID NO: 77 and with the same function as SEQ ID NO:77 or the amino acid sequence having at least 85% sequence identity with SEQ ID NO:77, and the amino acid sequence of the light chain variable region is SEQ ID NO:78, the amino acid sequence obtained by substituting, deleting or adding one or more amino acids of SEQ ID NO:78 and with the same function as SEQ ID NO:78 or the amino acid sequence having at least 85% sequence identity with SEQ ID NO:78.

In some specific embodiments, the present invention provides an anti-CLDN18.2 antibody or an antigen binding fragment thereof, wherein the amino acid sequence of the heavy chain variable region is SEQ ID NO:74, the amino acid sequence obtained by substituting, deleting or adding one or more amino acids of SEQ ID NO: 74 and with the same function as SEQ ID NO:74 or the amino acid sequence having at least 85% sequence identity with SEQ ID NO:74, and the amino acid sequence of the light chain variable region is SEQ ID NO:70, the amino acid sequence obtained by substituting, deleting or adding one or more amino acids of SEQ ID NO:70 and with the same function as SEQ ID NO:70 or the amino acid sequence having at least 85% sequence identity with SEQ ID NO:70.

In some specific embodiments, the present invention provides an anti-CLDN18.2 antibody or an antigen binding fragment thereof, wherein the amino acid sequence of the heavy chain variable region is SEQ ID NO:74, the amino acid sequence obtained by substituting, deleting or adding one or more amino acids of SEQ ID NO: 74 and with the same function as SEQ ID NO:74 or the amino acid sequence having at least 85% sequence identity with SEQ ID NO:74, and the amino acid sequence of the light chain variable region is SEQ ID NO:71, the amino acid sequence obtained by substituting, deleting or adding one or more amino acids of SEQ ID NO:71 and with the same function as SEQ ID NO:71 or the amino acid sequence having at least 85% sequence identity with SEQ ID NO:71.

In some specific embodiments, the present invention provides an anti-CLDN18.2 antibody or an antigen binding fragment thereof, wherein the amino acid sequence of the heavy chain variable region is SEQ ID NO:74, the amino acid sequence obtained by substituting, deleting or adding one or more amino acids of SEQ ID NO: 74 and with the same function as SEQ ID NO:74 or the amino acid sequence having at least 85% sequence identity with SEQ ID NO:74, and the amino acid sequence of the light chain variable region is SEQ ID NO:76, the amino acid sequence obtained by substituting, deleting or adding one or more amino acids of SEQ ID NO:76 and with the same function as SEQ ID NO:76 or the amino acid sequence having at least 85% sequence identity with SEQ ID NO:76.

In some specific embodiments, the present invention provides an anti-CLDN18.2 antibody or an antigen binding fragment thereof, wherein the amino acid sequence of the heavy chain variable region is SEQ ID NO:72, the amino acid sequence obtained by substituting, deleting or adding one or more amino acids of SEQ ID NO: 72 and with the same function as SEQ ID NO:72 or the amino acid sequence having at least 85% sequence identity with SEQ ID NO:72, and the amino acid sequence of the light chain variable region is SEQ ID NO:76, the amino acid sequence obtained by substituting, deleting or adding one or more amino acids of SEQ ID NO:76 and with the same function as SEQ ID NO:76 or the amino acid sequence having at least 85% sequence identity with SEQ ID NO:76.

In some specific embodiments, the present invention provides an anti-CLDN18.2 antibody or an antigen binding fragment thereof, wherein the amino acid sequence of the heavy chain variable region is SEQ ID NO:77, the amino acid sequence obtained by substituting, deleting or adding one or more amino acids of SEQ ID NO: 77 and with the same function as SEQ ID NO:77 or the amino acid sequence having at least 85% sequence identity with SEQ ID NO:77, and the amino acid sequence of the light chain variable region is SEQ ID NO:70, the amino acid sequence obtained by substituting, deleting or adding one or more amino acids of SEQ ID NO:70 and with the same function as SEQ ID NO:70 or the amino acid sequence having at least 85% sequence identity with SEQ ID NO:70.

In some specific embodiments, the present invention provides an anti-CLDN18.2 antibody or an antigen binding fragment thereof, wherein the amino acid sequence of the heavy chain variable region is SEQ ID NO:77, the amino acid sequence obtained by substituting, deleting or adding one or more amino acids of SEQ ID NO: 77 and with the same function as SEQ ID NO:77 or the amino acid sequence having at least 85% sequence identity with SEQ ID NO:77, and the amino acid sequence of the light chain variable region is SEQ ID NO:76, the amino acid sequence obtained by substituting, deleting or adding one or more amino acids of SEQ ID NO:76 and with the same function as SEQ ID NO:76 or the amino acid sequence having at least 85% sequence identity with SEQ ID NO:76.

In some specific embodiments, the present invention provides an anti-CLDN18.2 antibody or an antigen binding fragment thereof, wherein the amino acid sequence of the heavy chain variable region is SEQ ID NO:79, the amino acid sequence obtained by substituting, deleting or adding one or more amino acids of SEQ ID NO: 79 and with the same function as SEQ ID NO:79 or the amino acid sequence having at least 85% sequence identity with SEQ ID NO:79, and the amino acid sequence of the light chain variable region is SEQ ID NO:70, the amino acid sequence obtained by substituting, deleting or adding one or more amino acids of SEQ ID NO:70 and with the same function as SEQ ID NO:70 or the amino acid sequence having at least 85% sequence identity with SEQ ID NO:70.

In some specific embodiments, the present invention provides an anti-CLDN18.2 antibody or an antigen binding fragment thereof, wherein the amino acid sequence of the heavy chain variable region is SEQ ID NO:79, the amino acid sequence obtained by substituting, deleting or adding one or more amino acids of SEQ ID NO: 79 and with the same function as SEQ ID NO:79 or the amino acid sequence having at least 85% sequence identity with SEQ ID NO:79, and the amino acid sequence of the light chain variable region is SEQ ID NO:76, the amino acid sequence obtained by substituting, deleting or adding one or more amino acids of SEQ ID NO:76 and with the same function as SEQ ID NO:76 or the amino acid sequence having at least 85% sequence identity with SEQ ID NO:76.

In some specific embodiments, the present invention provides an anti-CLDN18.2 antibody or an antigen binding fragment thereof, wherein the amino acid sequence of the heavy chain variable region is SEQ ID NO:50, the amino acid sequence obtained by substituting, deleting or adding one or more amino acids of SEQ ID NO:50 and with the same function as SEQ ID NO:50 or the amino acid sequence having at least 85% sequence identity with SEQ ID NO:50, and the amino acid sequence of the light chain variable region is SEQ ID NO:80, the amino acid sequence obtained by substituting, deleting or adding one or more amino acids of SEQ ID NO:80 and with the same function as SEQ ID NO:80 or the amino acid sequence having at least 85% sequence identity with SEQ ID NO:80.

In some specific embodiments, the present invention provides an anti-CLDN18.2 antibody or an antigen binding fragment thereof, wherein the amino acid sequence of the heavy chain variable region is SEQ ID NO:50, the amino acid sequence obtained by substituting, deleting or adding one or more amino acids of SEQ ID NO:50 and with the same function as SEQ ID NO:50 or the amino acid sequence having at least 85% sequence identity with SEQ ID NO:50, and the amino acid sequence of the light chain variable region is SEQ ID NO:81, the amino acid sequence obtained by substituting, deleting or adding one or more amino acids of SEQ ID NO:81 and with the same function as SEQ ID NO:81 or the amino acid sequence having at least 85% sequence identity with SEQ ID NO:81.

In some specific embodiments, the present invention provides an anti-CLDN18.2 antibody or an antigen binding fragment thereof, wherein the amino acid sequence of the heavy chain variable region is SEQ ID NO:79, the amino acid sequence obtained by substituting, deleting or adding one or more amino acids of SEQ ID NO:79 and with the same function as SEQ ID NO:79 or the amino acid sequence having at least 85% sequence identity with SEQ ID NO:79, and the amino acid sequence of the light chain variable region is SEQ ID NO:80, the amino acid sequence obtained by substituting, deleting or adding one or more amino acids of SEQ ID NO:80 and with the same function as SEQ ID NO:80 or the amino acid sequence having at least 85% sequence identity with SEQ ID NO:80.

In some specific embodiments, the present invention provides an anti-CLDN18.2 antibody or an antigen binding fragment thereof, wherein the amino acid sequence of the heavy chain variable region is SEQ ID NO:79, the amino acid sequence obtained by substituting, deleting or adding one or more amino acids of SEQ ID NO:79 and with the same function as SEQ ID NO:79 or the amino acid sequence having at least 85% sequence identity with SEQ ID NO:79, and the amino acid sequence of the light chain variable region is SEQ ID NO:81, the amino acid sequence obtained by substituting, deleting or adding one or more amino acids of SEQ ID NO:81 and with the same function as SEQ ID NO:81 or the amino acid sequence having at least 85% sequence identity with SEQ ID NO:81.

In some specific embodiments, the present invention provides an anti-CLDN18.2 antibody or an antigen binding fragment thereof, wherein the antibody further contains a heavy chain constant region of human-derived IgG1 or variants thereof, and a light chain constant region of human-derived kappa chain or variants thereof.

In some specific embodiments, the present invention provides an anti-CLDN18.2 antibody or an antigen binding fragment thereof, wherein amino acid replacements in the heavy chain variable region with amino acid sequence shown in SEQ ID NO 79 can be made as follows:

(c1) K3Q, L19R, R42G, P45L, A49S, E73D, T78S, E82Q, S84N, S88A, M93V, A118S, the amino acid sequence after replacement is shown in SEQ ID NO 82;

(c2) K3Q, V5L, L19R, R42G, P45L, A49S, E73D, A75S, E82Q, S8N, S88A, M93V, A118S, the amino acid sequence after replacement is shown in SEQ ID NO 88.

And amino acid replacements in the light chain variable region with amino acid sequence shown in SEQ ID NO 81 can be made as follows:

(c3) DIE, S9P, S10T, T12S, V13L, T14S, A15P, K18R, K21L, P49A, K51L, G63S, V641, D66A, T69S, V84L, A86P, A106Q, L112I, the amino acid sequence after replacement is shown in SEQ ID NO 84;

(c4) S9D, T12A, T14S, A15L, K18R, V19A, M21I, S22N, T69S, V84L, L89V, A106Q, L112I, the amino acid sequence after replacement is shown in SEQ ID NO 86;

(c5) A9L, T12P, A15L, E17Q, K18P, V19A, T20S, M21I, K45R, P49S, K51R, T69S, T80K, S83R, Q85E, L89V, A90G, A106Q, L112I, the amino acid sequence after replacement is shown in SEQ ID NO 90.

In some specific embodiments, the present invention provides an anti-CLDN18.2 antibody or an antigen binding fragment thereof, wherein:

(1) the amino acid sequence of the heavy chain variable region is selected from:

(c1) an amino acid sequence shown in SEQ ID NO 82, SEQ ID NO 88;

(c2) an amino acid sequence obtained by substituting, deleting or adding one or more amino acids of the amino acid sequences shown in (c1), and with the same or similar function as the amino acid sequence shown in (c1); and

(c3) an amino acid sequence having at least 80% sequence identity with the amino acid sequence shown in (c1); and

(2) the amino acid sequence of the light chain variable region is selected from:

(c4) an amino acid sequence shown in SEQ ID NO 84, SEQ ID NO 86, SEQ ID NO 90;

(c5) an amino acid sequence obtained by substituting, deleting or adding one or more amino acids of the amino acid sequences shown in (c4), and with the same or similar function as the amino acid sequence shown in (c4); and

(c6) an amino acid sequence having at least 80% sequence identity with the amino acid sequence shown in (c4).

In some specific embodiments, the present invention provides an anti-CLDN18.2 antibody or an antigen binding fragment thereof, wherein the amino acid sequence of the heavy chain variable region is SEQ ID NO 82, the amino acid sequence obtained by substituting, deleting or adding one or more amino acids of SEQ ID NO 82 and with the same function as SEQ ID NO 82 or the amino acid sequence having at least 85% sequence identity with SEQ ID NO 82, and the amino acid sequence of the light chain variable region is SEQ ID NO 84, the amino acid sequence obtained by substituting, deleting or adding one or more amino acids of SEQ ID NO 84 and with the same function as SEQ ID NO 84 or the amino acid sequence having at least 85% sequence identity with SEQ ID NO 84.

In some specific embodiments, the present invention provides an anti-CLDN18.2 antibody or an antigen binding fragment thereof, wherein the amino acid sequence of the heavy chain variable region is SEQ ID NO 82, the amino acid sequence obtained by substituting, deleting or adding one or more amino acids of SEQ ID NO 82 and with the same function as SEQ ID NO 82 or the amino acid sequence having at least 85% sequence identity with SEQ ID NO 82, and the amino acid sequence of the light chain variable region is SEQ ID NO 86, the amino acid sequence obtained by substituting, deleting or adding one or more amino acids of SEQ ID NO 86 and with the same function as SEQ ID NO 86 or the amino acid sequence having at least 85% sequence identity with SEQ ID NO 86.

In some specific embodiments, the present invention provides an anti-CLDN18.2 antibody or an antigen binding fragment thereof, wherein the amino acid sequence of the heavy chain variable region is SEQ ID NO 88, the amino acid sequence obtained by substituting, deleting or adding one or more amino acids of SEQ ID NO 88 and with the same function as SEQ ID NO 88 or the amino acid sequence having at least 85% sequence identity with SEQ ID NO 88, and the amino acid sequence of the light chain variable region is SEQ ID NO 84, the amino acid sequence obtained by substituting, deleting or adding one or more amino acids of SEQ ID NO 84 and with the same function as SEQ ID NO 84 or the amino acid sequence having at least 85% sequence identity with SEQ ID NO 84.

In some specific embodiments, the present invention provides an anti-CLDN18.2 antibody or an antigen binding fragment thereof, wherein the amino acid sequence of the heavy chain variable region is SEQ ID NO 88, the amino acid sequence obtained by substituting, deleting or adding one or more amino acids of SEQ ID NO 88 and with the same function as SEQ ID NO 88 or the amino acid sequence having at least 85% sequence identity with SEQ ID NO 88, and the amino acid sequence of the light chain variable region is SEQ ID NO 86, the amino acid sequence obtained by substituting, deleting or adding one or more amino acids of SEQ ID NO 86 and with the same function as SEQ ID NO 86 or the amino acid sequence having at least 85% sequence identity with SEQ ID NO 86.

In some specific embodiments, the present invention provides an anti-CLDN18.2 antibody or an antigen binding fragment thereof, wherein the amino acid sequence of the heavy chain variable region is SEQ ID NO 82, the amino acid sequence obtained by substituting, deleting or adding one or more amino acids of SEQ ID NO 82 and with the same function as SEQ ID NO 82 or the amino acid sequence having at least 85% sequence identity with SEQ ID NO 82, and the amino acid sequence of the light chain variable region is SEQ ID NO 90, the amino acid sequence obtained by substituting, deleting or adding one or more amino acids of SEQ ID NO 90 and with the same function as SEQ ID NO 90 or the amino acid sequence having at least 85% sequence identity with SEQ ID NO 90.

In some specific embodiments, the present invention provides an anti-CLDN18.2 antibody or an antigen binding fragment thereof, wherein the amino acid sequence of the heavy chain variable region is SEQ ID NO 88, the amino acid sequence obtained by substituting, deleting or adding one or more amino acids of SEQ ID NO 88 and with the same function as SEQ ID NO 88 or the amino acid sequence having at least 85% sequence identity with SEQ ID NO 88, and the amino acid sequence of the light chain variable region is SEQ ID NO 90, the amino acid sequence obtained by substituting, deleting or adding one or more amino acids of SEQ ID NO 90 and with the same function as SEQ ID NO 90 or the amino acid sequence having at least 85% sequence identity with SEQ ID NO 90.

In some specific embodiments, the present invention provides an anti-CLDN18.2 antibody or an antigen binding fragment thereof, wherein the amino acid sequence of the heavy chain is SEQ ID NO 92, the amino acid sequence obtained by substituting, deleting or adding one or more amino acids of SEQ ID NO 92 and with the same function as SEQ ID NO 92 or the amino acid sequence having at least 85% sequence identity with SEQ ID NO 92, and the amino acid sequence of the light chain is SEQ ID NO 94, the amino acid sequence obtained by substituting, deleting or adding one or more amino acids of SEQ ID NO 94 and with the same function as SEQ ID NO 94 or the amino acid sequence having at least 85% sequence identity with SEQ ID NO 94.

In some specific embodiments, the present invention provides an anti-CLDN18.2 antibody or an antigen binding fragment thereof, wherein the amino acid sequence of the heavy chain is SEQ ID NO 96, the amino acid sequence obtained by substituting, deleting or adding one or more amino acids of SEQ ID NO 96 and with the same function as SEQ ID NO 96 or the amino acid sequence having at least 85% sequence identity with SEQ ID NO 96, and the amino acid sequence of the light chain is SEQ ID NO 98, the amino acid sequence obtained by substituting, deleting or adding one or more amino acids of SEQ ID NO 98 and with the same function as SEQ ID NO 98 or the amino acid sequence having at least 85% sequence identity with SEQ ID NO 98.

Another aspect of the present invention provides an isolated nucleotide, which encodes the anti-CLDN18.2 antibody or the antigen binding fragment thereof of the present invention.

In some specific embodiments, the present invention provides nucleotide sequences encoding the anti-CLDN18.2 antibody or the antigen binding fragment thereof of the present invention, wherein,

(1) the nucleotide sequence encoding the amino acid sequence of the heavy chain is shown in SEQ ID NO 93 or SEQ ID NO 97;

(2) the nucleotide sequence encoding the amino acid sequence of the light chain is shown in SEQ ID NO 95 or SEQ ID NO 99.

In some specific embodiments, the present invention provides nucleotide sequences encoding the anti-CLDN18.2 antibody or the antigen binding fragment thereof of the present invention, wherein,

(1) the nucleotide sequence encoding the amino acid sequence SEQ ID NO 92 of the heavy chain is shown in SEQ ID NO 93; and

(2) the nucleotide sequence encoding the amino acid sequence SEQ ID NO 94 of the light chain is shown in SEQ ID NO 95.

In some specific embodiments, the present invention provides nucleotide sequences encoding the anti-CLDN18.2 antibody or the antigen binding fragment thereof of the present invention, wherein,

(1) the nucleotide sequence encoding the amino acid sequence SEQ ID NO 96 of the heavy chain is shown in SEQ ID NO 97; and

(2) the nucleotide sequence encoding the amino acid sequence SEQ ID NO 98 of the light chain is shown in SEQ ID NO 99.

Another aspect of the present invention provides an expression vector, which expresses the anti-CLDN18.2 antibody or the antigen binding fragment thereof of the present invention. The expression vector according to the present invention comprises the isolated nucleotide molecule of the present invention.

Another aspect of the present invention provides a host cell transfected with the expression vector as described above.

In some embodiments, the host cell according to the present invention is selected from a prokaryotic cell and a eukaryotic cell. In some embodiments, the host cell is bacteria, preferably Escherichia coli. In another preferred embodiment, the host cell is a mammalian cell.

Another aspect of the present invention provides a method for preparing the anti-CLDN18.2 antibody or the antigen binding fragment thereof of the present invention, which comprises steps of expressing the antibody in the host cell and isolating the antibody from the host cell.

Another aspect of the present invention provides a pharmaceutical composition, which comprises the anti-CLDN18.2 humanized antibody or the antigen binding fragment thereof of the present invention and a pharmaceutically acceptable carrier. In some embodiments, the present invention provides a pharmaceutical composition, which comprises the anti-CLDN18.2 humanized antibody or the antigen binding fragment thereof of the present invention, as well as other active components, such as other antibodies, targeting drugs and the like. In some embodiments, the pharmaceutically acceptable carrier is selected from antioxidants, polypeptides, proteins, hydrophilic polymers, amino acids, sugars, chelating agents, sugar alcohols, ions, and surfactants. In one specific embodiment, the pharmaceutically acceptable carrier is a buffered aqueous solution. In another specific embodiment, the pharmaceutically acceptable carrier is in the form of liposomes.

The anti-CLDN18.2 humanized antibody or the antigen binding fragment thereof of the present invention can be mixed with a pharmaceutically acceptable carrier, diluent or excipient to prepare a pharmaceutical preparation suitable for oral or parenteral administration. Methods of administration include, but are not limited to oral, intradermal, intramuscular, intraperitoneal, intravenous, intracerebral, intraocular, intratracheal, subcutaneous, and intranasal routes. The preparation can be administered by any route, for example, by infusion or bolus injection, by the route of absorption through the epithelium or skin mucosa (for example, oral mucosa or rectum, etc.). Administration can be systemic or local. The preparation can be prepared by methods known in the art, and comprises carriers, diluents or excipients conventionally used in the field of pharmaceutical preparations.

Another aspect of the present invention provides a method for therapy targeting CLDN18.2, including administering the anti-CLDN18.2 antibody or the antigen binding fragment thereof of the present invention or the pharmaceutical composition of the present invention to an individual in need thereof.

Another aspect of the present invention provides the use of the anti-CLDN18.2 antibody or the antigen binding fragment thereof of the present invention or the pharmaceutical composition of the present invention in the preparation of a drug targeting CLDN18.2. In some embodiments, the drug targeting CLDN18.2 is used to treat gastric cancer, esophageal cancer, pancreatic cancer, lung cancer, ovarian cancer, colon cancer, liver cancer, head and neck cancer, and gallbladder cancer. In some embodiments, the present invention provides the use of the aforementioned anti-CLDN18.2 antibody or the antigen binding fragment thereof or the pharmaceutical composition of the present invention in the preparation of an anti-tumor drug, preferably, the tumor is selected from gastric cancer, esophageal cancer, pancreatic cancer, lung cancer, ovarian cancer, colon cancer, liver cancer, head and neck cancer and gallbladder cancer.

The anti-CLDN18.2 antibody or the antigen binding fragment thereof provided by the present invention has a significant anti-tumor effect, high affinity, strong in-vitro ADCC cytotoxicity against tumor cells, and has significant technical advantages.

### Definition

Unless otherwise defined, the scientific and technical terms used herein have a meaning that is commonly understood by those skilled in the art. The nomenclatures and techniques used in the cell and tissue culture, molecular biology, and protein and oligo- or poly-nucleotide chemistry and hybridization described herein are well known and commonly used in the art. For recombinant DNA, oligonucleotide synthesis, and tissue culture and transfection (such as electroporation, lipofection), standard techniques are used. The enzymatic reaction and purification techniques are carried out according to the manufacturer's manual or methods commonly used in the art or described herein. The aforementioned techniques and methods are generally used as described in multiple comprehensive and relatively specific documents well known in the art and cited and discussed in the specification. See, for example, Sambrook et al., Molecular Cloning: A Laboratory Manual (2nd edition, Cold Spring Harbor Laboratory Press, Cold Spring Harbor, N.Y. (1989)). The nomenclatures and laboratory methods and techniques used in analytical chemistry, synthetic organic chemistry, and medical and pharmaceutical chemistry described herein are well known and commonly used in the art.

In the present invention, the term “at least 80% sequence identity” refers to at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity. In the present invention, the term “at least 85% sequence identity” means at least 85%, 86%, 87%, 88%, 89%, 90%, 91%, 92%, 93%, 94%, 95%, 96%, 97%, 98%, 99%, or 100% sequence identity. In some preferred embodiments, the sequence identity described in the present invention may be at least 90%, 91%, 92%, 93%, 94%, 95%, 96%, 97%, 98%, 99%, or 100%. Sequence comparison and determination of percent identity between two sequences can be performed by the BLASTN/BLASTP algorithm on the National Center For Biotechnology Institute website.

In an antibody molecule, the three hypervariable regions of the light chain and the three hypervariable regions of the heavy chain are arranged relative to each other in the three-dimensional space to form an antigen binding surface. The antigen binding surface is complementary to the three-dimensional surface of the bound antigen, and the three hypervariable regions of each heavy chain and light chain are referred to as “complementarity determining regions” or “CDRs”. The assignment of amino acids to each domain is defined according to Kabat “Sequences of Proteins of Immunological Interest” (National Institutes of Health, Bethesda, Md. (1987 and 1991)) or Chothia and Lesk, J. Mol. Biol. 196:901-917 (1987), Chothia et al., Nature 342:878-883 (1989).

The “antigen binding fragment” described in the present invention refers to Fab fragments, Fab′ fragments, and F(ab′)2 fragments which have antigen binding activity, and Fv fragments and scFv fragments which bind to the human Claudin18.2. The Fv fragment contains the heavy chain variable region and the light chain variable region of an antibody, but no constant region, and is the smallest antibody fragment with all the antigen binding sites. Generally, the Fv antibody also comprises a polypeptide linker between the VH and VL domains, and can form the structure required for the antigen binding. Different linkers can also be used to connect the two antibody variable regions to form a polypeptide chain, referred to as single-chain antibody or single-chain Fv (scFv).

The antibody described in the present invention refers to an immunoglobulin molecule or an immunologically active part thereof, that is, a molecule comprising an antigen binding site that specifically binds to (immunologically reacts with) an antigen. “Specific binding” refers to that an antibody reacts with one or more epitopes of an antigen but does not react with other polypeptides or binds to other polypeptides with a very low affinity (Kd>10-6). Antibodies include, but are not limited to, polyclonal antibodies, monoclonal antibodies, chimeric antibodies, dAbs (domain antibodies), single chain antibodies, Fab, Fab′ and F(ab′)2 fragments, Fv, scFv and Fab expression libraries. Monoclonal antibodies (mAbs) are antibodies obtained from a single clonal cell strain, and the cell strain is not limited to eukaryotic, prokaryotic or phage clonal cell strain. Monoclonal antibodies or antigen binding fragments thereof can be recombinantly obtained, for example, by using hybridoma technology, recombination technology, phage display technology, and synthesis technology such as CDR grafting or other existing technologies.

The “murine-derived antibody” described in the present invention is a monoclonal antibody against the human CLDN18.2 prepared according to the knowledge and skills in the art. During the preparation, test subjects are injected with CLDN18.2 antigen, and then hybridomas expressing antibodies with desired sequences or functional characteristics are isolated.

The “chimeric antibody” described in the present invention is an antibody formed by fusing the variable region of a murine-derived antibody with the constant region of a human antibody, which can reduce immune responses induced by the murine-derived antibody. To construct the chimeric antibodies, it is necessary to construct hybridomas secreting murine-derived specific monoclonal antibodies first, and then clone the variable region gene from the mouse hybridoma cells, and then clone the constant region gene of the human antibody as needed, connect the mouse variable region gene to the human constant region gene to form the chimeric gene which is then inserted into an expression vector, and finally express the chimeric antibody molecule in a eukaryotic industrial system or a prokaryotic industrial system.

The “humanized antibody” described in the present invention is also referred to as CDR grafted antibody, which is an antibody produced by grafting the mouse CDR sequence into the human antibody variable region framework (FR). Such variable region framework sequences can be obtained from public DNA databases or published references, for example, from the ImMunoGeneTics (IMGT) website http://imgt.cines.fr or from the Journal of Immunoglobulin, 2001ISBN012441351.

### BRIEF DESCRIPTION OF THE DRAWINGS

FIG. 1 is the binding of the chimeric antibodies to CLDN18.2 cells, wherein the horizontal axis is the antibody concentration in ng/ml; the vertical axis is the OD value at 450 nm.

FIG. 2 is the binding of the chimeric antibodies to CLDN18.1 cells, wherein the horizontal axis is the antibody concentration in ng/ml; the vertical axis is the OD value at 450 nm.

FIG. 3 is the binding of the phage Fabs to the CLDN18.2 protein, wherein the horizontal axis is the phage titer; the vertical axis is the OD value at 450 nm.

FIG. 4 is the binding of the humanized antibodies to CHO-K1-CLDN18.2 cells, wherein the horizontal axis is the antibody concentration in mg/ml; the vertical axis is the average fluorescence intensity MFI of binding to the cells.

FIG. 5 is the binding of the humanized antibodies to CHO-K1-CLDN18.1 cells, wherein the horizontal axis is the antibody concentration in mg/ml; the vertical axis is the average fluorescence intensity MFI of binding to the cells.

FIG. 6 is the ADCC activity of the humanized antibodies.

### DETAILED DESCRIPTION OF THE INVENTION

The following representative examples are used to better illustrate the present invention, rather than to limit the protection scope of the present invention. The experimental methods without indicated conditions in the following examples are generally carried out according to conventional conditions, such as the antibody technology experimental manual, molecular cloning manual or the like of Cold Spring Harbor, or according to the conditions recommended by the raw material or product manufacturers. Materials and reagents used in the examples are all commercially available unless otherwise specified.

### Example 1 Preparation of the Positive Control Antibody

1. Preparation of the Positive Control Antibody IMAB362-Similar-hG1:

According to the 175D10 clone (IMAB362) antibody sequence provided by the patent application WO2007/059997 (i.e., SEQ ID NO 11 and SEQ ID NO 12 in the present disclosure), Nanjing GenScript Biotechnology Co., Ltd. was entrusted to perform codon optimization and synthesize it in vector pcDNA3.1(+). The constructed plasmids include pcDNA3.1-hG1 and pcDNA3.1-hK. expiCHO cells were co-transfected with the plasmids at a ratio of 1:1 for transient expression, cultured for 4 days at 110 rpm, 37° C., 8% CO₂, transferred to a 32° C. shaker to further culture for 8-10 days. After centrifugation at 4500 rpm for 25 minutes, the protein supernatant obtained was purified by ProteinA affinity chromatography as follows: the AKTA chromatography system and columns were cleaned with 0.1 M NaOH solution for 30 minutes, rinsed clean with ultrapure water; equilibrated with 10 column volumes of 20 mM sodium phosphate buffer of pH 7.4; the flow rate was set to 2 ml/min, samples were loaded; the columns were cleaned with 10 column volumes of the equilibration buffer; eluted with 3-5 column volumes of 50 mM acetate buffer of pH 3.4, the eluted protein was collected; concentrated via ultrafiltration to displace the antibody in 20 mM phosphate buffer of pH 7.0 for storage.

Results of the SDS-PAGE gel electrophoresis show that: the molecular weight of the positive antibody IMAB362-similar-hG1 is about 155 KD, the purity by SDS-PAGE is greater than 90%.

2. Preparation of Murine-Derived Positive Control Antibody IMAB362-Similar-mG2a:

That is, a combined antibody sequence of the positive antibody variable region sequence SEQ ID NO 13 and mouse IgG2a constant region sequence SEQ ID NO 14. It was synthesized in vector pcDNA3.1(+) after codon optimization. The constructed plasmids include pcDNA3.1-mG2a and pcDNA3.1-mK. expiCHO cells were co-transfected with the plasmids at a ratio of 1:1 for transient expression of the positive antibody proteins. The protein supernatant obtained was purified by ProteinA affinity chromatography as follows: the AKTA chromatography system and columns were cleaned with 0.1 M NaOH solution for 30 minutes, rinsed clean with ultrapure water; equilibrated with 10 column volumes of 20 mM sodium phosphate buffer of pH 7.4; the flow rate was set to 2 ml/min, samples were loaded; the columns were cleaned with 10 column volumes of the equilibration buffer; eluted with 3-5 column volumes of 50 mM acetate buffer of pH 3.4, the eluted protein was collected; concentrated via ultrafiltration to displace the antibody in 20 mM phosphate buffer of pH 7.0 for storage.

Results of the SDS-PAGE gel electrophoresis show that: non-denaturing electrophoresis of the positive antibody IMAB362-similar-mG2a shows its protein molecular weight is about 155 KD, the purity by SDS-PAGE is greater than 90%.

### Example 2 Synthesis of the CLDN18.2 Antigen Gene and Construction of the Expression Vector

The sequence design of the fusion protein expressing the human CLDN18.2 antigen is shown in Table 1 below:

TABLE 1
CLDN18.2 sequence design

| | Sequence | | Amino acid | Nucleotide |
|---|---|---|---|---|
| No. | name | Sequence meaning | sequence | sequence |
| 1 | 18A2-loop1 | Encoding the sequence | SEQ ID | SEQ ID |
| | | of extracellular loop 1 | NO 2 | NO 1 |
| | | of CLDN18.2 | | |
| 2 | 18A2-TM- | Encoding the sequence | SEQ ID | SEQ ID |
| | Loop1 | of extracellular loop 1 | NO 4 | NO 3 |
| | | comprising the | | |
| | | transmembrane domain | | |
| | | of CLDN18.2 | | |
| 3 | CpG | Unmethylated cytosine | | SEQ ID |
| | | guanine dinucleotide | | NO 5 |
| | | palindrome | | |
| 4 | CLDN18.2- | Encoding the | SEQ ID | SEQ ID |
| | FL | full-length protein | NO 8 | NO 6 |
| | | sequence of the human | | SEQ ID |
| | | CLDN18.2 | | NO 7 |
| 5 | CLDN18.1- | Encoding the | SEQ ID | SEQ ID |
| | FL | full-length protein | NO 10 | NO 9 |
| | | sequence of the human | | |
| | | CLDN18.1 | | |

Eukaryotic codon optimization was carried out for the fusion protein sequence of the human CLDN18.2 antigen designed above, and restriction sites such as NotI and tPA signal peptide sequences were added to the 5′ end of the gene, while restriction sites such as XhoI and Myc-His fusion protein tag were added to the 3′ end of the gene. The above SEQ ID NO 1, 3, 5, 6 sequences were synthesized and constructed into the pcDNA3.1(+) vector to obtain DNA plasmids expressing the human CLDN18.2 antigen. The plasmids include: pcDNA3.1-loop1, pcDNA3.1-TML, pcDNA3.1-CpG-loop1 and pcDNA3.1-CLDN18.2-FL.

The above SEQ ID NO 7, 9 sequences were optimized and then synthesized by Bao Biological Engineering (Dalian) Co., Ltd. (hereinafter referred to as Bao Biological Company), and constructed into the lentiviral vector pLVX-NS-ZsGreen (Bao Biological Company). The constructed lentiviral plasmids pLVX-CLDN18.2-ZsGreen and pLVX-CLDN18.1-ZsGreen were used to further construct a cell line stably expressing HEK293-CLDN18.2 fusion green fluorescent protein, and to construct the stable cell line expressing HEK293-CLDN18.1 fusion green fluorescent protein.

The amplified plasmids were used for restriction enzyme digestion identification, and the identification results show that the digested band of interest is consistent with the expected size, indicating that the construction is correct.

### Example 3 Expression of the CLDN18.2 Antigen Protein

1. Transient Transfection with Plasmid DNA In Vitro

HEK293T cells (Institute of Biochemistry and Cell Biology, Chinese Academy of Sciences, hereinafter referred to as Shanghai Institute of Cell Biology) were transiently transfected with the plasmid amplified in Example 2 in vitro, and analyzed at the protein level. The detailed experimental steps are as follows: one day before transfection, HEK293T cells were plated in six-well plates at a seeding density of 3×10⁵ cells/cm² and cultured (culture medium composition: DMEM culture medium with 10% fetal bovine serum+1% penicillin-streptomycin) overnight to make the cells fully attached to the plate on the day of transfection, and to make the cells reach 70%-90% confluency (the size of the six-well plate is 9 cm² per well, full confluency means about 2-2.5×10⁶ cells/well); the DNA plasmids were mixed with PEI cationic transfection reagent (sigma) or liposomes (Lipo3000, Thermo) to prepare the DNA plasmids transfection complex, added to cells and let stand at room temperature for 5-20 minutes, incubated at 37° C., 5% CO₂ for 4-5 hours; complete culture medium containing serum was added to the six-well plate and cultured for 48-72 hours to transiently express the exogenous gene in eukaryotic cells in vitro.

2. Western Blot Immunoblotting Hybridization

HEK293T cells post transient transfection were collected, and the target protein was hybridized with rabbit anti-human Claudin18 antibody (Thermo, Cat: 700178) at room temperature for 2 hours; washed 3 times with PBST, hybridized with alkaline phosphatase-conjugated goat anti-rabbit IgG (Thermo, Cat: 31346) at room temperature for 1 hour; Western Blot immunoblotted to identify the expression level of the target protein: with Lipo liposome transfection system, the plasmids can successfully express the target protein CLDN18.2-loop1 and CLDN18.2-TM-loop1, with molecular weights of 11 KD and 14 KD, respectively, pcDNA3.1-CLDN18.2-FL plasmid successfully expressed the target protein CLDN18.2 with a molecular weight of 29 KD.

### Example 4 Preparation of the Mouse Hybridomas

1. Animal Immunization:

The plasmid DNAs were extracted and prepared according to the manual of EndoFree Plasmid Maxi Kit from QIAGEN, including pcDNA3.1-loop1 (hereinafter referred to as Loop1), pcDNA3.1-TM-Loop1 (hereinafter referred to as TML), pcDNA3.1-CpG-loop1 (hereinafter referred to as Loop1-C). The plasmids were grouped according to the following Table 2 for mouse immunization, wherein the SJL mice were purchased from Beijing Vital River Laboratory Animal Technology Co., Ltd., and the BALB/c mice and the C57 mice were purchased from Huafukang Biological Products Center, Xuanwu District, Nanjing. For immunization, refer to the method described in Zhiwei Chen et al. (Z. Chen., et al., J Clin Invest. 123(6): 2629-2642 (2013)), with appropriate modifications, detailed as follows: DNA plasmids were injected into the left and right hind limb muscles of the mouse at two points respectively through the In Vivo Gene Importer (Shanghai Taresa Biotechnology Co., Ltd.), and the same amount of DNA plasmids was injected at each point, with an injection volume of 50 μl. Immediately after the injection, the electrode of the importer was used to input electrical pulse stimulation on the injection sites of the mouse, the voltage was set to 36V, and 6 pulses in total. Electrical pulse stimulation can promote the uptake of DNA plasmids by mouse muscle cells and increase the level of mouse immune responses to DNA, especially the level of humoral immune responses. DNA immunization was injected once every 2 weeks for a total of three immunizations. Seven days after the second and the third immunizations, blood was collected from the fundus venous plexus of the mouse for flow cytometry analysis, and when a clear CLDN18 positive signal was detected in serum antibodies, HEK293-CLDN18.2 cells were used as antigens for the mouse, prepared as cell suspension of 5×10⁷ cells/ml, injected with 100 μl (5×10⁶ cells) into the intraperitoneal cavity of the mouse to boost immunity.

TABLE 2
Mouse immunization groups

| | | Number | DNA prime immunization | | | Cell boost |
|---|---|---|---|---|---|---|
| Group | Type of mice | of mice | Week 0 | Week 2 | Week 4 | immunization |

| | | | | |
|---|---|---|---|---|
| 1 | BALB/c, female, | 3 | Loop1, 50 μg per animal | 3-4 days before |
| 2 | SPF, 6-8 weeks | 3 | Loop1-C, 50 μg per animal | the fusion of |
| 3 | | 3 | TML, 50 μg per animal | splenocytes, 5 × 10⁶ |
| 4 | SJL, female, | 3 | Loop1, 100 μg per animal | HEK293-CLDN18.2 |
| 5 | SPF, 4-6 weeks | 4 | Loop1-C, 100 μg per animal | cells per animal |
| 6 | | 3 | TML, 100 μg per animal | were injected |
| 7 | C57, female, | 3 | Loop1, 25 μg per animal | intraperitoneally |
| 8 | SPF, 6-8 weeks | 3 | Loop 1-C, 25 μg per animal | |
| 9 | | 3 | TML, 25 μg per animal | |

2. FACS Detection of the Immune Serum:

One week after the second and the third DNA immunizations, blood was collected from the fundus venous plexus of the mouse to prepare the immune serum, respectively. The serum was diluted 1:50 in volume using the blocking solution (2% BSA in PBS). Human gastric cancer cells NUGC4 (Nanjing Co-bioer Biosciences Co., Ltd.) were resuscitated and cultured in advance, in 1640 culture medium containing 10% fetal bovine serum, 37° C., 5% CO₂. When the cells were in good condition and the cell growth confluence was 70-80%, gastric cancer cell digestion solution (CHI Scientific, Inc.) was used to digest the cells at 37° C. for 15 minutes. Complete 1640 culture medium was used to terminate the digestion, centrifuged at 1000 rpm for 5 minutes, the supernatant was discarded. Appropriate amount of the blocking solution was added to resuspend the cells, the cell density was adjusted to 1×10⁶ cells/ml. 100 μl of NUGC4 cells were mixed with 100 μl of diluted mouse serum, incubated at 4° C. for 1 hour; washed twice with Cell Stain Buffer (Biolegend), centrifuged at 1000 rpm for 5 minutes at each time, and the supernatant was discarded. 100 μl of PE-conjugated goat anti-mouse antibodies (Biolegend) were added, incubated at 4° C. for 1 hour; washed twice with Cell Stain Buffer (Biolegend), centrifuged at 1000 rpm for 5 minutes at each time, and the supernatant was discarded. 300 μl of Cell Stain Buffer was added to resuspend the cells, detected with the flow cytometry analyzer.

After the second DNA immunization, an obvious deviation of the positive signal was detected in mouse number #0103, indicating that this mouse produced specific antibody responses against CLDN18. After the third DNA immunization, positive signals were detected in all mice, wherein the CLDN18 positive rates in mice with numbers #0101, #0102, #0103, #0203, #0301, and #0303 were greater than 50%, indicating these mice produced obvious antibody immune responses, and there were relatively high levels of anti-CLDN18 antibodies in their serum after the third DNA immunization.

3. Preparation of the Mouse Hybridoma Cells

Preparation of mouse myeloma cell suspension: mouse myeloma cells SP2/0 were resuscitated two weeks in advance, cultured and passaged in 1640 culture medium containing 10% fetal bovine serum at 37° C., 5% CO₂. On the day of fusion, the status of SP2/0 cells was observed. The cell confluence of SP2/0 cells was 70%-80% by microscopic examination, therefore it can be determined that the cells were in the logarithmic growth phase. SP2/0 cells were collected by centrifugation at 300 g for 5 minutes, washed once with 20 ml of PBS, 1000 rpm, 5 min, the supernatant was discarded; cells were resuspended with an appropriate amount of 1640 culture medium, and the concentration of SP2/0 cells was adjusted to 1×10⁷ cells/ml for later use.

Preparation of mouse feeder cell suspension: the day before fusion, 20 healthy Balb/c mice aged 10-12 weeks were sacrificed through cervical dislocation, the mouse abdominal skin was cut open using sterile surgical scissors, a small cut was made across the abdomen first, and then the chest war cut longitudinally to expose the mouse abdomen. Collection of peritoneal macrophages: the mouse abdomen was disinfected with alcohol cotton ball first, and then the mouse was injected intraperitoneally with 5 ml of 1640 basal culture medium using a syringe, the mouse abdomen was massaged gently for 5 minutes, then the fluid in the abdominal cavity was aspirated with the syringe. Repeated once, 3 ml of 1640 basal culture medium was injected and then aspirated (or the peritoneum was cut open, then the remaining macrophages were aspirated using pasteur pipettes). The collected macrophages were centrifuged at 1500 rpm for 10 minutes, the supernatant was discarded, the cells were resuspended using 10 ml (1640 culture medium containing 10% fetal bovine serum) for later use.

Preparation of mouse spleen lymphocyte suspension: 3-4 days after cell boost immunization, splenocytes of the immunized mice were taken to fuse with SP2/0. The mice were sacrificed through cervical dislocation, the peritoneum was cut open sterilely, the spleen was taken out and the surrounding connective tissue was peeled off. Mouse splenocytes were grinded and then filtered with a 70 μm sieve into a 50 ml centrifuge tube pre-filled with 15 ml of 1640 culture medium to prepare the single cell suspension; 1500 rpm, 5 min, washed with 1640 culture medium, the supernatant was discarded; 4 ml of red blood cell lysis solution (Sigma) was added to lyse red blood cells for about 2 min; an equal volume of 1640 culture medium was added, centrifuged at 1500 rpm for 5 min, the upper layer of red blood cells was discarded; resuspended using 20 ml of PBS, washed twice, centrifuged at 1500 rpm for 5 min. After the second wash, the cells were resuspended with 15 ml of PBS, and if there were residual agglomerated connective tissue at this time, all the cells can be filtered through sieves and transferred to a new 50 ml centrifuge tube; the spleen lymphocytes were counted, resuspended with an appropriate amount of 1640 culture medium, the splenocytes were adjusted to 1×10⁷ cells/ml.

1×10⁸ spleen cells were mixed with SP2/0 cells in the ratio of 1:1, centrifuged at 1500 rpm for 5 minutes, the supernatant was discarded. Cells were resuspended and mixed well with BTX electrofusion buffer, and electrofusion operations were performed according to the manual of the BTX electrofusion instrument. The fused cells were allowed to stand at room temperature for 30 minutes, and 1640 selective culture medium containing 1×HAT and 10% fetal bovine serum was added, the feeder cell suspension prepared in advance was also added, plated to a total of 200 96-well plates, incubated at 37° C., 5% CO₂ for 5-7 days. Then, the fused hybridoma cell clones were cultured with refreshed culture medium containing 1×HT.

### Example 5 Preparation of the Positive Cell Line

a. Construction of CHO-K1-CLDN18.2 Stable Cell Line

CHO-K1 cells (Shanghai Institute of Cell Biology) were resuscitated one week in advance, and cultured in DME/F12 complete culture medium containing 10% fetal bovine serum at 37° C., 5% CO₂. The plasmid used to transfect the cells was pcDNA3.1-CLDN18.2-FL (see SEQ ID NO 6 for the synthetic sequence). On the day of transfection, the growth density of CHO-K1 cells reached 80%-90%. According to the manual of the Biorad Gene Pulser Xcell electroporation instrument, the plasmids were transfected into CHO-K1 cells, and all the transfected cells were transferred to petri dishes with pre-heated complete culture medium, let stand at 37° C., 5% CO₂ overnight to culture. Next day, G418 (Sigma) was added at a final concentration of 1 mg/ml for resistance screening for 3-4 days, the CLDN18.2 expression level of cells was detected after passaging for at least 5 generations using complete culture medium containing G418 resistance to stabilize the clone. The results show that the transfected cells stably express the human Claudin18.2 after passaging for 5 generations compared with CHO-K1 empty cells, by using the positive antibody IMAB362-similar-hG1 (final concentration of 10 μg/ml) to be incubated with the cells and detected with flow cytometry.

b. Construction CHO-K1-CLDN18.1 Stable Cell Line Using the pLVX-CLDN18.1-ZsGreen plasmid as a template, primer sequences SEQ ID NO 15 and SEQ ID NO 16 were designed, the PCR system was set up according to manual of PrimeStar (Takara), the PCR program was set to denature at 98° C. for 30 seconds, anneal at 60° C. for 30 seconds, extend at 72° C. for 1 minute and amplify for 30 cycles to obtain the CLDN18.1-FL target gene fragment (see SEQ ID NO 17). The target gene and pcDNA3.1(+) vector were digested with the two enzymes BamHI and EcoRI, ligated with T4 ligase (Takara) at 16° C. overnight. The ligation product was transformed into E. coli, and the plasmid pcDNA3.1-CLDN18.1 was extracted with miniprep kit. The plasmid was electrotransfected into CHO-K1 cells according to the steps above, and the cells were cultured and passaged in culture medium with G418 resistance, Western Blot results show that the transfected cells can be stained positive with anti-CLDN18 antibody (Thermo), while FACS analyses show that the transfected cells exhibit negative fluorescent signals after incubating with the CLDN18.2 positive antibody IMAB362-similar-hG1 at a concentration of 10 μg/ml. It indicates that the transfected CLDN18.1 cells are successfully constructed.

### Example 6 Antibody Screen of the Mother Clones of Hybridomas

The first round of primary screening was carried out for the mother clones of hybridoma cells obtained after the fusion, and the screening methods include cellular ELISA and FACS to detect supernatant of the hybridoma cell culture, respectively, from which the mother clones of the CLDN18 positive hybridomas were screened. The CLDN18 positive clones in the primary screen were amplified, then the second round of re-screening was carried out, from which mother clones of hybridomas showing CLDN18.2 positive and CLDN18.1 negative in FACS were screened. After two rounds of cellular ELISA and FACS screening of hybridoma antibodies, a total of 9 mother clones secreting CLDN18.2-specific antibodies were screened out of hundreds of mother clones, which can positively bind with HEK293-CLDN18.2 cells, while the binding to HEK293-CLDN18.1 cells was negative.

### Example 7 Culture and Screening of Subclones

1. Culture of Subclones

Monoclonal clones were prepared from the hybridoma cells obtained from screenings in Example 6. Hybridoma cells were resuspended in 24-well plates to prepare the cell suspension. Cells were counted for each clone, the final cell concentration was adjusted to 1×10⁴ cells/ml using 1640 complete culture medium; 60 μl of cell suspension was added to 140 μl of 1640 complete culture medium to achieve a total of 200 μl, respectively, mixed well; the semi-solid culture medium D (StemCell) stored at −20° C. was taken out one day in advance, thawed at 4° C., and placed at 37° C. for 1 hour before use. 2.5 ml of semi-solid culture medium was taken and mixed thoroughly with 200 μl of cell suspension, let stand at room temperature for 15 minutes; the well mixed cell suspension in semi-solid culture medium was transferred to 6-well cell culture plates (Corning), the inoculation quantity being 600 cells per well, an appropriate amount of sterile water was added around the plates, placed at 37° C., 5% CO₂ for 5-7 days. When the cells in the plates grew into dense spherical colonies of monoclonal cells, then they were aspirated using 10 μl pipette tips and transferred to 200 μl of 1640 culture medium containing 1×HT, 10% CloneEasy and 10% fetal bovine serum, cell suspension was thoroughly mixed and incubated at 37° C., 5% CO₂ for 2-3 days.

2. Screening of Subclones

To identify the CLDN18.2 specificity of antibodies in the cell culture supernatant of the subclones, the screening method used was cellular ELISA to detect the positive binding of supernatant antibodies to CLDN18.2 and CLDN18.1 stably transfected cells. The detailed protocol was as follows:

HEK293-CLDN18.1 and HEK293-CLDN18.2 cells were cultured separately with DMEM complete culture medium containing 10% fetal bovine serum at 37° C., 5% CO₂. When the cells grew to 80%-90% confluency, they were digested with 0.5 mM EDTA-PBS non-trypsin digestion solution at room temperature, and centrifuged at 1000 rpm for 5 minutes, the supernatant was discarded. Resuspended in DMEM complete culture medium, and the cell concentration was adjusted to 6×10⁵ cells/ml. Cells were plated on 96-well cell plates (Corning) pre-coated with polylysine, 100 μl per well, incubated at 37° C., 5% CO₂ for 24 hours, making cells evenly and completely plated on the bottom of the well. The culture supernatant was aspirated, and the cells were fixed with 4% paraformaldehyde at room temperature for 20 minutes, washed 3 times with 200 μl PBS, 2 minutes at each time. The well plates were blocked with 200 μl of 3% BSA-PBS at 37° C. for 1 hour. 100p of subclone culture supernatant was taken and added into the well plate, incubated at 37° C. for 1 hour, washed 5 times with PBS. Incubation with HRP-anti mouse IgG+IgM (Jackson) diluted 1:10000 in blocking solution was performed at 37° C. for 1 hour followed by washing 5 times with PBS. 100 μl of TMB one-component color developing solution was added, protected from light at room temperature for 5-10 minutes, terminated with 2M sulfuric acid, and OD450 was read on the microplate reader. The results are shown in Table 3, subclones of which the hybridoma culture supernatant show strong positive binding to CLDN18.2 (that is, the OD value is 50% higher than the OD value of the positive control) include: S3F10C5, S5H3A6, S5H3B6, S5H3D6, S6F10E7, S6H9B8, S6H9C8, C40B10D9, C42B12D10 and C58B11E11. Among these subclones, S5H3A6, S5H3B6, S5H3D6, C40B10D9, C42B12D10 and C58B11E11 show negative binding to CLDN18.1 cells.

TABLE 3
Results of subclone screening using cellular ELISA

| | Subclone | OD450 | OD450 |
|---|---|---|---|
| No. | No. | (CLDN18.2) | (CLDN18.1) |
| 1 | mG2a | 1.286 | 0.168 |
| | (positive | | |
| | control) | | |
| 2 | B4B10G3 | 0.343 | 0.200 |
| 3 | S3F10B5 | 0.615 | 0.198 |
| 4 | S3F10C5 | 0.948 | 0.278 |
| 5 | S3F10E5 | 0.504 | 0.213 |
| 6 | S5H3A6 | 1.928 | 0.192 |
| 7 | S5H3B6 | 1.379 | 0.152 |
| 8 | S5H3D6 | 1.636 | 0.166 |
| 9 | S6F10E7 | 0.818 | 0.350 |
| 10 | S6F10H7 | 0.452 | 0.205 |
| 11 | S6H9B8 | 2.353 | 1.495 |
| 12 | S6H9C8 | 2.165 | 1.303 |
| 13 | C40B10D9 | 0.664 | 0.114 |
| 14 | C42B12D10 | 0.760 | 0.172 |
| 15 | C58B11E11 | 0.671 | 0.197 |

Clonal expansion was carried out for subclones B4B10G3, S5H3B6, S6H9B8, C40B10D9, C42B12D10 and C58B11 E11. Cells in the 96-well plate were resuspended with 200 μL pipettes, all the cell suspension was transferred into the 24-well cell plate (Corning), 1 ml of 1640 culture medium containing 2% CloneEasy, 10% fetal bovine serum was added, placed in a 37° C., 5% CO₂ incubator to incubate for 3-4 days.

### Example 8 Identification of Antibody Subtypes

The subclone cell culture supernatant to be detected was added to identify the subtypes of the mouse antibodies according to the manual of the mouse antibody subtype identification kit SBA Clonotyping System (SouthernBiotech). The identification results are as follows:

Subclone B4B10G3, the subtype of the heavy chain constant region of the antibody is the murine IgG2b; the subtype of the light chain constant region of the antibody is the murine Kappa chain;

Subclone S5H3B6, the subtype of the heavy chain constant region of the antibody is the murine IgM; the subtype of the light chain constant region of the antibody is the murine Kappa chain;

Subclone S6H9B8, the subtype of the heavy chain constant region of the antibody is the murine IgG3; the subtype of the light chain constant region of the antibody is the murine Kappa chain;

Subclone C58B11E11, the subtype of the heavy chain constant region of the antibody is the murine IgG2c; the subtype of the light chain constant region of the antibody is the murine Kappa chain;

Subclone C40B10D9, the subtype of the heavy chain constant region of the antibody is the murine IgM; the subtype of the light chain constant region of the antibody is the murine Kappa chain;

Subclone C42B12D10, the subtype of the heavy chain constant region of the antibody is the murine IgM; the subtype of the light chain constant region of the antibody is the murine Kappa chain;

### Example 9 Sequencing of the Variable Region of the Antibody

Monoclonal hybridoma cells in the 24-well plate were collected, adjusted to 1×10⁶ cells/ml, 1 ml in total, centrifuged at 1000 rpm for 5 minutes, the supernatant was discarded. Miniprep of total RNA of cells was carried out according the manual of RNA extraction kit (Cat:#74134) from QIAGEN, NanoDrop nucleic acid quantitative analyzer was used to determine the RNA concentration. Next, 2 μg of total RNA was reverse transcribed into cDNA according to the Takera Reverse Transcription Kit (Cat:#6110A), stored at −80° C. for later use. Using cDNA as template, the PCR systems were prepared and the PCR instrument programs were set up for sequencing the antibody light and heavy chains respectively according to manual of PremixTaq enzyme from Takara. The primers used in the PCR system were synthesized by Suzhou GENEWIZ Inc., and detailed primer sequences are as follows:

heavy chain sequencing primer VH-Fs including:

VH-F1 (comprising primer sequences SEQ ID NO 18 and SEQ ID NO 19), VH-F2 (comprising primer sequences SEQ ID NO 20, SEQ ID NO 21, SEQ ID NO 22, and primer sequences SEQ ID NO 23, SEQ ID NO 24, SEQ ID NO 25), VH-F3 (primer sequence SEQ ID NO 26) heavy chain sequencing primer VH-Rs including:

VH-R1 (primer sequence SEQ ID NO 27), VH-R2 (primer sequence SEQ ID NO 28)

light chain sequencing primer VL-Fs including:

VL-F1 (primer sequence SEQ ID NO 29), VL-F2 (comprising primer sequences SEQ ID NO 30 and SEQ ID NO 31), VL-F3 (comprising primer sequences SEQ ID NO 32, SEQ ID NO 33, SEQ ID NO 34)

light chain sequencing primer VL-Rs including:

VL-R (primer sequence SEQ ID NO 35)

The PCR products obtained by PCR amplification were entrusted to Suzhou GENEWIZ Inc. for sequencing, and the sequencing results are shown in Table 4.

TABLE 4
Table of nucleotide sequences of subclones

| | Nucleotide sequence | |
|---|---|---|
| | Heavy chain | Light chain |
| Subclone | variable region | variable region |
| B4B10G3 | SEQ ID NO 36 | SEQ ID NO 37 |
| S5H3B6 | SEQ ID NO 38 | SEQ ID NO 39 |
| S6H9B8 | SEQ ID NO 40 | SEQ ID NO 41 |
| C58B11E11 | SEQ ID NO 42 | SEQ ID NO 43 |
| C40B10D9 | SEQ ID NO 44 | SEQ ID NO 45 |
| C42B12D10 | SEQ ID NO 46 | SEQ ID NO 47 |

### Example 10 Preparation of the Chimeric Antibodies

According to the variable region sequences of the murine-derived antibodies obtained in Example 9, the constant region of the human IgG1 and the variable region of the mouse antibodies were combined to construct the human-murine chimeric antibodies through gene synthesis to further study the antibody affinity and antibody properties.

a. Synthesis of the Human-Murine Chimeric Antibody Genes

Suzhou GENEWIZ Biotechnology Co., Ltd. was entrusted to synthesize optimized chimeric antibody genes and construct them into the eukaryotic expression vector pcDNA3.1(+), and the chimeric antibodies were named as 1CHI, 3CHI, 7CHI, 17CHI, 18CHI, 19CHI, respectively. The antibody variable region sequence corresponding to each chimeric antibody is shown in Table 5 below:

TABLE 5
Sequences of the chimeric antibodies

| | | Amino acid sequence | | Nucleotide sequence | |
|---|---|---|---|---|---|
| | | Heavy | Light | Heavy | Light |
| | | chain | chain | chain | chain |
| Monoclonal | Chimeric | variable | variable | variable | variable |
| antibody | antibody | region | region | region | region |
| B4B10G3 | 1CHI | SEQ ID | SEQ ID | SEQ ID | SEQ ID |
| | | NO 48 | NO 49 | NO 36 | NO 37 |
| S5H3B6 | 3CHI | SEQ ID | SEQ ID | SEQ ID | SEQ ID |
| | | NO 50 | NO 51 | NO 52 | NO 53 |
| S6H9B8 | 7CHI | SEQ ID | SEQ ID | SEQ ID | SEQ ID |
| | | NO 54 | NO 55 | NO 56 | NO 57 |
| C58B11E11 | 17CHI | SEQ ID | SEQ ID | SEQ ID | SEQ ID |
| | | NO 58 | NO 59 | NO 60 | NO 61 |
| C40B10D9 | 18CHI | SEQ ID | SEQ ID | SEQ ID | SEQ ID |
| | | NO 62 | NO 63 | NO 64 | NO 65 |
| C42B12D10 | 19CHI | SEQ ID | SEQ ID | SEQ ID | SEQ ID |
| | | NO 66 | NO 67 | NO 68 | NO 69 |

b. Expression and Protein Purification of the Chimeric Antibodies

Plasmids containing the light and heavy chain genes of the above chimeric antibodies were transiently transfected into HEK293E cells (Shanghai Institute of Cell Biology) in the ratio of 1:1. The cells are shaking cultured for 7 days with shaking speed of 110 rpm, 37° C., 8% CO₂; centrifuged at 4500 rpm for 20 minutes; and the supernatant protein was harvested. Purification by ProteinA affinity chromatography is performed as follows: the AKTA chromatography system and columns were cleaned with 0.1 M NaOH solution for 30 minutes, rinsed clean with ultrapure water; equilibrated with 10 column volumes of 20 mM sodium phosphate buffer of pH 7.4; the flow rate was set to 2 ml/min, samples were loaded; the columns were cleaned with 10 column volumes of the equilibration buffer; eluted with 3-5 column volumes of 50 mM acetate buffer of pH 3.4, the eluted protein was collected; concentrated via ultrafiltration to displace the antibodies in 50 mM acetate buffer of pH 5.55 for storage.

Among them, the SDS-PAGE gel electrophoresis of the chimeric antibodies 3CHI, 18CHI and 19CHI shows that the purity of the chimeric antibodies 3CHI, 18CHI and 19CHI by SDS-PAGE are all greater than 90%.

### Example 11 Determination of the Specific Binding of the Chimeric Antibodies to CLDN18.2

HEK293-CLDN18.1 and HEK293-CLDN18.2 cells were resuscitated two weeks in advance, cultured with DMEM complete culture medium containing 10% fetal bovine serum at 37° C., 5% CO₂. When the cells grew to 80%-90% confluency, they were digested with 0.5 mM EDTA-PBS (Sigma) at room temperature, centrifuged at 1000 rpm for 5 minutes, the supernatant was discarded. Cells were resuspended in DMEM complete culture medium, the cell concentration was adjusted to 6×10⁵ cells/ml. Cells were plated on 96-well cell plates (Corning) pre-coated with polylysine at 2.5 μg/well, 100 μl per well, incubated at 37° C., 5% CO₂ for 24 hours, making cells evenly and completely plated on the bottom of the well. The supernatant was aspirated, and the cells were fixed with 4% paraformaldehyde at room temperature for 20 minutes, washed 3 times with 200 μl PBS, 2 minutes at each time. The well plates were blocked with 200 μl of 3% BSA-PBS at 37° C. for 1 hour. Each chimeric antibody was diluted with 3% BSA-PBS to a concentration of 6 μg/ml, and diluted in a 6-fold gradient to a total of 7 concentrations. At the same time, IMAB362-similar-hG1 diluted in the same concentration gradient as the positive control, and human IgG1, kappa isotype control antibody (hereinafter referred to as Isotype, provided by CrownBio (Taicang) Co., Ltd.) as the negative control were added to HEK293-CLDN18.2 and HEK293-CLDN18.1 cell plates, respectively. The cells were incubated at 37° C. for 1 hour, washed 5 times with PBS. Incubation with HRP-anti human IgG (Jackson) diluted 1:10000 in blocking solution was performed at 37° C. for 1 hour followed by washing 5 times with PBS. 100 μl of TMB one-component color developing solution was added, protected from light at room temperature for 5-10 minutes, terminated with 50 μl of 2M sulfuric acid, OD450 was read on the microplate reader. Data processing: the original data was put into GraphPad 5.0 software for plotting and calculation. The results are shown in Table 6.

TABLE 6
Determination of the specific binding of the chimeric
antibodies to CLDN18.2

| Antibody No. | EC50 (nM) |
|---|---|
| Positive control (IMAB362-similar-hG1) | 0.09 |
| Negative control (Isotype) | N.A |
| 1CHI | 0.55 |
| 3CHI | 0.08 |
| 17CHI | 2.44 |
| 18CHI | 1.75 |
| 19CHI | 29.90 |

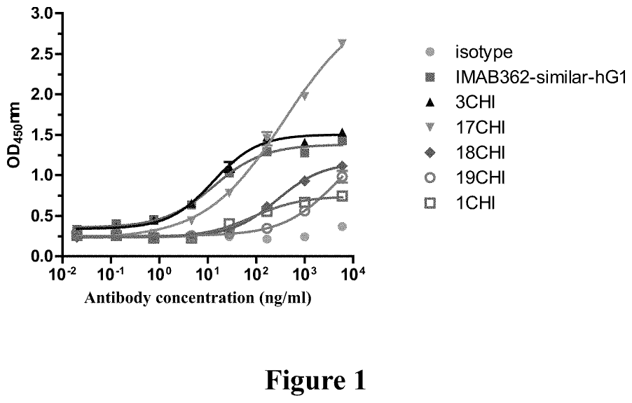

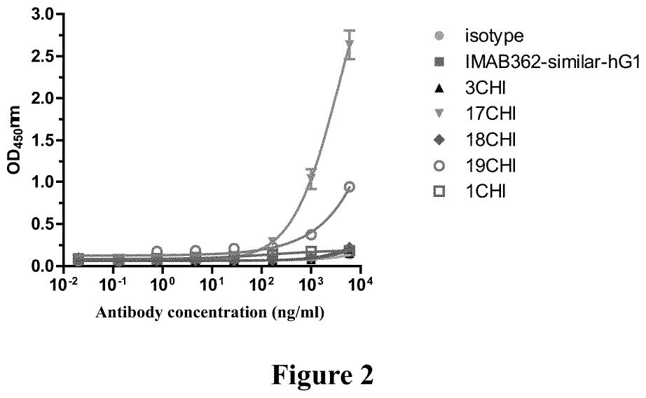

The binding of each chimeric antibody to CLDN18.2 cells and CLDN18.1 cells is shown in FIG. 1 and FIG. 2, wherein the Isotype is a negative control and IMAB362-similar-hG1 is a positive control. The results show that the binding curve of each chimeric antibody is concentration gradient dependent. Among them, 17CHI exhibits strong positive binding to both CLDN18.2 and CLDN18.1, whereas 19CHI exhibits weak positive binding to CLDN18.2 and CLDN18.1, while 1CHI, 3CHI and 18CHI antibodies can specifically bind to CLDN18.2 without binding to CLDN18.1. At the same time, among the anti-CLDN18.2 specific antibodies, the EC50 value of 3CHI antibody is relatively low, and similar to the EC50 value of the positive control antibody IMAB362-similar-hG1, showing relatively strong activity of specifically binding to CLDN18.2.

### Example 12 Affinity Maturation of the Chimeric Antibody

The chimeric antibody 3CHI with the closest affinity to the positive control antibody in Example 11 was selected for affinity maturation and screening in vitro. The phage Fab antibody library was mainly used for screening to obtain antibodies that can maintain the CLDN18.2 specificity while have increased affinity.

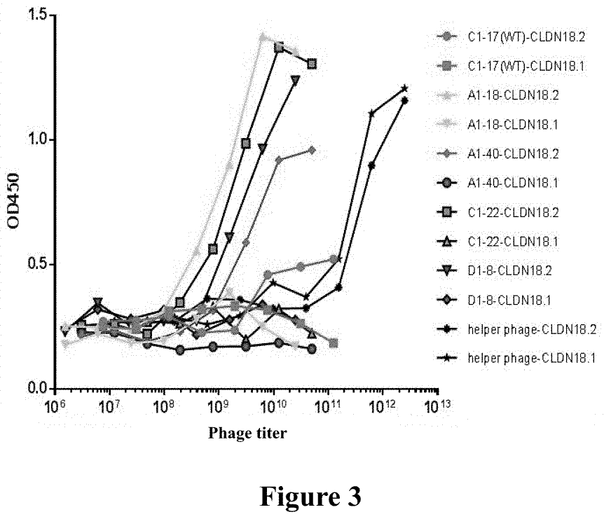

The results of the phage screening are shown in FIG. 3, phage antibodies C1-22, A1-18, or the like all exhibit higher CLDN18.2 binding activity than the original 3CHI phage antibody (WT). CDR sequences with amino acid site mutations were obtained after sequencing the phages, and these mutation sites were recombined to construct chimeric antibodies with the constant region of human IgG1. After renumbering and naming the chimeric antibodies derived from phages, whether the 30 chimeric antibodies above specifically bind to CLDN18.2 was identified by cellular ELISA. The results are shown in Table 7, there are 18 specific antibodies with negative binding to CLDN18.1 and positive binding to CLDN18.2 in the table, and their amino acid sequences are shown in Table 11.

TABLE 7
Screening results of the phage-derived chimeric antibodies

| Identification | |
|---|---|
| content | Antibody name |
| CLDN18.1 positive and | 310CHI, 311CHI, 314CHI, 316CHI, 317CHI, |
| CLDN18.2 positive | 318CHI, 320CHI, 323CHI, 325CHI, 326CHI, |
| | 327CHI, 329CHI |
| CLDN18.1 negative and | 301CHI, 302CHI, 303CHI, 304CHI, 305CHI, |
| CLDN18.2 positive | 306CHI, 307CHI, 308CHI, 309CHI, 312CHI, |
| | 313CHI, 315CHI, 319CHI, 321CHI, 322CHI, |
| | 324CHI, 328CHI, 330CHI |

TABLE 8
List of amino acid sequences of the specific chimeric antibodies

| | Antibody | Heavy chain | Light chain |
|---|---|---|---|
| No. | name | variable region | variable region |
| 1 | 301CHI | SEQ ID NO 50 | SEQ ID NO 70 |
| 2 | 302CHI | SEQ ID NO 50 | SEQ ID NO 71 |
| 3 | 303CHI | SEQ ID NO 72 | SEQ ID NO 73 |
| 4 | 304CHI | SEQ ID NO 74 | SEQ ID NO 75 |
| 5 | 305CHI | SEQ ID NO 50 | SEQ ID NO 76 |
| 6 | 306CHI | SEQ ID NO 77 | SEQ ID NO 78 |
| 7 | 307CHI | SEQ ID NO 74 | SEQ ID NO 70 |
| 8 | 308CHI | SEQ ID NO 74 | SEQ ID NO 71 |
| 9 | 309CHI | SEQ ID NO 74 | SEQ ID NO 76 |
| 10 | 312CHI | SEQ ID NO 72 | SEQ ID NO 76 |
| 11 | 313CHI | SEQ ID NO 77 | SEQ ID NO 70 |
| 12 | 315CHI | SEQ ID NO 77 | SEQ ID NO 76 |
| 13 | 319CHI | SEQ ID NO 79 | SEQ ID NO 70 |
| 14 | 321CHI | SEQ ID NO 79 | SEQ ID NO 76 |
| 15 | 322CHI | SEQ ID NO 50 | SEQ ID NO 80 |
| 16 | 324CHI | SEQ ID NO 50 | SEQ ID NO 81 |
| 17 | 328CHI | SEQ ID NO 79 | SEQ ID NO 80 |
| 18 | 330CHI | SEQ ID NO 79 | SEQ ID NO 81 |

Human gastric cancer cells NUGC4 and stably transfected cells CHO-K1-CLDN18.1 (prepared in Example 5) were used respectively, to verify the specificity and affinity of the anti-CLDN18.2 antibodies above at the cellular level by FAGS technology. The results are shown in Table 9. After antibody affinity maturation and screening in vitro, each antibody can bind to the CLDN18.2 target on NUGC4 cells, but not to CHO-K1-CLDN18.1 cells, as compared with the original 3CHI antibody. According to the level of antibody affinity indicated by the MFI (Mean Fluorescence Intensity) of FACS, the 330CHI antibody shows a significant increase in its affinity for targeting NUGC4 cells, as compared with the positive control antibody IMAB362-similar-hG1.

TABLE 9
FACS detection results of the affinity-matured antibodies

| Antibody No. | MFI (CLDN18.2) | MFI (CLDN18.1) |
|---|---|---|
| Isotype | 93 | 219 |
| IMAB362-similar-hG1 | 2179 | 59 |
| 3CHI | 2509 | 76 |
| 17CHI | N.A | 14455 |
| 301CHI | 44420 | 114 |
| 303CHI | 38241 | 85 |
| 305CHI | 38913 | 68 |
| 309CHI | 42495 | 78 |
| 315CHI | 42301 | 65 |
| 321CHI | 46327 | 299 |
| 322CHI | 44777 | 57 |
| 324CHI | 36030 | 248 |
| 330CHI | 63666 | 49 |

The affinity of the anti-CLDN18.2 antibodies above were verified at the protein level by the Fortebio molecular interaction instrument: first, the antibody solution and the antigen dilution were prepared by diluting the chimeric antibody with SD buffer (PBS solution containing 0.02% Tween20 and 0.1% BSA) to a final concentration of 5 μg/ml; and by diluting the antigen protein Claudin18.2-His with SD buffer in a 4-fold concentration gradient to make the concentrations 10 μg/ml, 2.5 μg/ml, 0.625 μg/ml, 0 μg/ml, respectively. Next, antibodies were immobilized by the AHC sensor, and then combined with different concentrations of antigens, and determination of the affinity was performed according to the manual of Octet RED96.

The results show that the equilibrium dissociation constant (KD) of the binding of the positive control antibody IMAB362-similar-hG1 to CLDN18.2 protein at 37° C. was 7.14 nM, and the KD values of 330CHI and 305CHI antibodies were 0.84 nM and 0.87 nM, respectively, indicating a significantly increase by an order of magnitude compared with the positive control (see Table 10).

TABLE 10
Affinity test results of the affinity-matured antibodies

| | Antibody | Type of | | | |
|---|---|---|---|---|---|
| No. | name | sensor | KD (M) | Kon (M⁻¹ · s⁻¹) | Kdis (s⁻¹) |
| 1 | IMAB362- | AHC | 7.14 × 10⁻⁹ | 5.59 × 10⁴ | 3.99 × 10⁻⁴ |
| | similar-hG1 | | | | |
| 2 | 330CHI | | 8.40 × 10⁻¹⁰ | 1.39 × 10⁵ | 1.17 × 10⁻⁴ |
| 3 | 305CHI | | 8.65 × 10⁻¹⁰ | 5.13 × 10⁴ | 4.44 × 10⁻⁵ |

### Example 13 Determination of the In-Vitro ADCC Activities of the Chimeric Antibodies

The day before the determination experiment, CHO-K1-CLDN18.2 cells (prepared in Example 5) were digested to be resuspended in complete culture medium, the cells were counted, and the density was adjusted to 1×10⁵ cells/ml, inoculated on 96-well plates in 100 μl/well, incubated overnight in a 37° C., 5% CO₂ incubator; on the day of the test, the phenol red-free 1640 culture medium (Gibco) was incubated to 37° C., and the antibody was diluted in a 5-fold gradient; 96-well plates plated with CHO-K1-CLDN18.2 were taken out, culture medium was discarded, 25 μl of phenol red-free 1640 was added to each well, the diluted antibody was added to the sample wells, making the final antibody concentrations being 10 μg/ml, 2 μg/ml, 0.4 μg/ml, 0.08 μg/ml, 0.016 μg/ml and 0.0032 μg/ml, respectively; equal amounts of IMAB362-similar-hG1 and human IgG1 Isotype control antibody (CrownBio) were added to the control wells, incubated in a 37° C., 5% CO₂ incubator for 1 h;

Jurkat cells stably expressing the human CD16A protein and NFAT reporter gene protein (hereinafter referred to as Jurkat-CD16A/NF) were used as the effector cells, transferred to centrifuge tubes, centrifuged at 1000 rpm for 5 min at room temperature, the supernatant was discarded; resuspended and washed with 5 ml of phenol red-free 1640 culture medium once, centrifuged at 1000 rpm for 5 min, the supernatant was discarded; the cells were resuspended in phenol red-free 1640, and the cell density was adjusted to 8×10⁶ cells/ml;

2×10⁵ Jurkat-CD16A/NF cells were added to the well plate according to an effector-to-target ratio of 20:1 (that is, the ratio of the number of the effector cells to the target cells is 20:1), centrifuged at 200 g for 2 minutes to make the effector cells fully contacted with the target cells; incubated in a 37° C., 5% CO₂ incubator for 5-6 hours; 30 minutes before the detection, cell plates were recovered to room temperature, detection reagents were added according the manual of the Bio-Turbo Firefly Luciferase Assay Kit (Jiangsu Riogene Biotechnology Co., Ltd.), the supernatant to be detected was transferred to white flat-bottom 96-well plates, and fluorescent signals of the well plates were detected with Thermo Floskan Ascent FL fluorescence microplate reader. The original data was put into Graphpad 5.0 software for calculation and analysis, the results are shown in Table 11.

TABLE 11
Determination results of the in-vitro ADCC activities
of the chimeric antibodies

| Group | EC50 (nM) | Top |
|---|---|---|
| Isotype | 13.48 | 6190 |
| IMAB362-similar-hG1 | 0.72 | 215966 |
| 3CHI | 0.54 | 211611 |
| 305CHI | 0.28 | 215996 |
| 330CHI | 0.22 | 214800 |

Compared with the Isotype negative control, the fluorescent signal of each chimeric antibody was dose-dependent with the concentration gradient. In the experimental group, the EC50 of the chimeric antibody 330CHI is 0.22 nM and the EC50 of the chimeric antibody 305CHI is 0.28 nM, indicating the ADCC EC50 activity of the chimeric antibodies was more than 3 times higher than that of the positive control antibody IMAB362-similar-hG1 (0.72 nM). As can be seen, the chimeric antibodies have improved cell binding activity and affinity against CLDN18.2, as well as effectively improved in-vitro ADCC activity effects of the antibodies.

### Example 14 Humanization of the Antibody

The sequence of antibody 330CHI modified by affinity maturation in vitro in Example 12 was optimized by humanization. Sequence optimization by humanization and identification of antibody physical and chemical properties were entrusted to Hangzhou Healsun Biopharm Co., Ltd. In the sequence optimization by humanization, the source of the murine sequence of the variable region of the 3CHI antibody and 330CHI antibody was confirmed through the immune gene database (IMGT), and through homologous comparison, the FR region of the heavy chain variable region sequence of the 3CHI antibody and 330CHI antibody was derived from the mouse antibody IGHV5-15*01; while the FR sequence of the light chain variable region of the antibody was derived from the mouse antibody IGKV8-19*01. The CDR region of the mouse antibody and the FR region of the human antibody were recombined to form the humanized antibody, wherein the similarities between the used light and heavy chain templates of human-derived antibodies and the corresponding variable region sequences of mouse antibodies were 65% or more, see Table 12 for the source of the human-derived antibodies. The names and sequences of the final humanized antibodies are shown in Table 13.

TABLE 12
Design information of the humanized sequences

| | | | |
|---|---|---|---|
| Source of the | | Whether or not the | Recombined |
| human-derived | | CDR length is the same | humanized |
| sequence | Similarity | as the mouse antibody | sequence |

Humanization design of the heavy chain

| | | | |
|---|---|---|---|
| IGHV3-48*01 | 77.55% | Yes | SEQ ID NO 82 |
| IGHV3-23*01 | 75.51% | Yes | SEQ ID NO 88 |

Humanization design of the light chain

| | | | |
|---|---|---|---|
| IGKV4-1*01 | 81.91% | Yes | SEQ ID NO 84 |
| IGKV3-7*01 | 65.96% | No | SEQ ID NO 86 |
| IGKV2-30*01 | 64.89% | No | SEQ ID NO 90 |

TABLE 13
Sequence listing of the humanized antibodies

| | | Amino acid sequence | | Nucleotide sequence | |
|---|---|---|---|---|---|
| | | Heavy chain | Light chain | Heavy chain | Light chain |
| | Antibody | variable | variable | variable | variable |
| No. | name | region | region | region | region |
| 1 | Hu330-1 | SEQ ID NO 82 | SEQ ID NO 84 | SEQ ID NO 83 | SEQ ID NO 85 |
| 2 | Hu330-2 | SEQ ID NO 82 | SEQ ID NO 86 | SEQ ID NO 83 | SEQ ID NO 87 |
| 3 | Hu330-3 | SEQ ID NO 88 | SEQ ID NO 84 | SEQ ID NO 89 | SEQ ID NO 85 |
| 4 | Hu330-4 | SEQ ID NO 88 | SEQ ID NO 86 | SEQ ID NO 89 | SEQ ID NO 87 |
| 5 | Hu330-5 | SEQ ID NO 82 | SEQ ID NO 90 | SEQ ID NO 83 | SEQ ID NO 91 |
| 6 | Hu330-6 | SEQ ID NO 88 | SEQ ID NO 90 | SEQ ID NO 89 | SEQ ID NO 91 |

Plasmid vectors comprising genes of the humanized antibody light or heavy chain were co-transfected into HEK293E cells at a ratio of 1:1, cultured with shaking at 110 rpm, 37° C., 8% CO₂ for 7 days. The cells were centrifuged at 4500 rpm for 20 minutes, the supernatant protein was harvested. Purification by ProteinA affinity chromatography was performed as follows: the AKTA chromatography system and columns were cleaned with 0.1 M NaOH solution for 30 minutes, rinsed clean with ultrapure water; equilibrated with 10 column volumes of 20 mM sodium phosphate buffer of pH 7.4; the flow rate was set to 2 ml/min, samples were loaded; the columns were cleaned with 10 column volumes of the equilibration buffer; eluted with 3-5 column volumes of 50 mM acetate buffer of pH 3.4, the eluted protein was collected; concentrated via ultrafiltration to displace the antibody in 20 mM phosphate buffer of pH 6.5 for storage. The purity of the purified antibody was determined by size exclusion high performance liquid chromatography (SEC-HPLC), and the monomer protein purity of humanized antibodies Hu330-1, Hu330-2 and Hu330-4 all reach more than 95%, among them, for Hu330-1 as an example, the molecular weight of the antibody heavy chain is 48.72 KD and the molecular weight of the light chain is 24.21 KD.

### Example 15 FACS Detection of the Specificity and Affinity of the Binding of the Humanized Antibodies to Human CLDN18.2 at the Cellular Level

CHO-K1-CLDN18.2 cells and CHO-K1-CLDN18.1 cells (prepared in Example 5) were digested with gastric cancer cell digestion solution at 37° C. for 15 minutes, respectively; terminated with complete culture medium, centrifuged at 1000 rpm for 5 minutes, the supernatant was discarded; the cells were resuspended with 2% fetal bovine serum-PBS diluent, the cells were adjusted to 1×10⁶ cells/ml, 100 μl of cell suspension was added to each well of the 96-well U-shaped-bottom plate; 2% fetal bovine serum-PBS was used to dilute the humanized antibody and the control antibody to final concentrations of 10 μg/ml, 2 μg/ml, 0.4 μg/ml, 0.08 μg/ml and 0.016 μg/ml, respectively, mixed well with the cells in equal volume, incubated at 4° C. for 1 hour; centrifuged at 1000 rpm for 5 minutes, the supernatant was discarded; 200 μl Cell Stain Buffer (Biolegend) was added to each well to wash twice, centrifuged at 1000 rpm for 5 minutes at each time and the supernatant was discarded; 100 μl of PE-anti human Fc antibody (Biolegend) was added, incubated at 4° C. for 1 hour; washed with Cell Stain Buffer (Biolegend) twice, centrifuged at 1000 rpm for 5 minutes at each time and the supernatant was discarded. 300 μl Cell Stain Buffer was added to resuspend the cells, and detected by BD FACS Jazz flow cytometry analyzer.

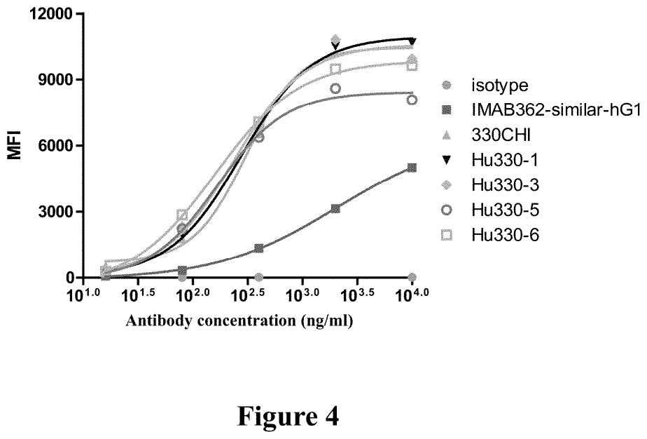

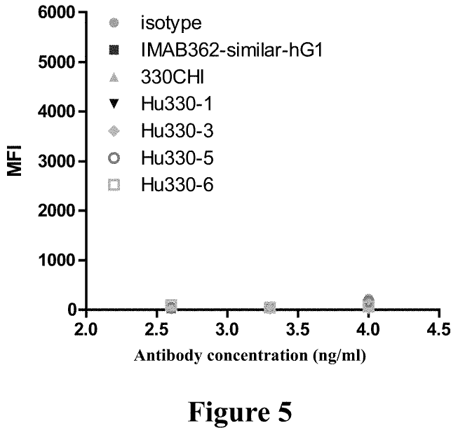

The results are shown in FIG. 4 and FIG. 5, humanized antibodies Hu330-1, Hu330-3, Hu330-5, and Hu330-6 all showed specific binding to CLDN18.2, while the binding to CLDN18.1 cells was negative. Among them, the binding curve of the humanized antibody Hu330-1 almost completely overlapped with that of the chimeric antibody 330CHI, indicating that after the antibody humanization, its affinity was not significantly different from that of the chimeric antibody, basically maintained at the same level. The values of the relative equilibrium dissociation constant Kd of antibodies calculated according to the Graphpad fitting curve are shown in Table 14. The Kd value of the humanized antibody Hu330-1 is 1.7 nM, which is significantly lower than that of the positive control antibody (28.7 nM), indicating the binding activity of the humanized antibody to cells expressing CLDN18.2 is about 15 times higher than that of the positive control antibody IMAB362-similar-hG1.

TABLE 14
Fitting results of the relative equilibrium dissociation constant Kd
of the humanized antibody determined by FACS

| No. | Antibody name | Kd (nM) | Hill slope | Bmax (MFI) |
|---|---|---|---|---|
| 1 | IMAB362-similar-hG1 | 28.7 | 4.816 | 8224 |
| 2 | 330CHI | 1.6 | 7.566 | 10760 |
| 3 | Hu330-1 | 1.7 | 6.781 | 11321 |

### Example 16 Fortebio Detection of the Specificity and Affinity of the Binding of the Humanized Antibody to Human CLDN18.2 at the Protein Level

Another method is to verify the affinity of the humanized antibody above at the protein level. The antibody was immobilized by the AHC sensor, and combined with different concentrations of antigens, and affinity determination was performed according to the manual of Octet RED96. The results are shown in Table 15. The equilibrium dissociation constant (KD) of the binding of the humanized antibody Hu330-1 to CLDN18.2 protein at 37° C. is 5.26 nM, indicating that the antibody affinity significantly is increased by about 18 times compared with the positive control.

TABLE 15
Relative equilibrium dissociation constant KD value of the
humanized antibody determined by Fortebio

| No. | Antibody name | KD (M) | Kon (M⁻¹ · s⁻¹) | Kdis (s⁻¹) |
|---|---|---|---|---|
| 1 | IMAB362- | 9.44 × 10⁻⁹ | 2.35 × 10⁵ | 2.22 × 10⁻³ |
| | similar-hG1 | | | |
| 2 | 330CHI | 2.49 × 10⁻¹⁰ | 1.30 × 10⁵ | 3.23 × 10⁻⁵ |
| 3 | Hu330-1 | 5.26 × 10⁻¹⁰ | 1.75 × 10⁵ | 9.22 × 10⁻⁵ |

### Example 17 Determination of the In-Vitro ADCC Activities of the Humanized Antibodies by Luciferase Labeling

CHO-K1-CLDN18.2 cells (prepared in Example 5) were used as target cells, the cell density was adjusted to 1×10⁵ cells/ml, inoculated 100 μl/well into 96-well plates, incubated overnight in a 37° C., 5% CO₂ incubator; the humanized antibody was diluted in a 5-fold gradient, making the final antibody concentrations 10 μg/ml, 2 μg/ml, 0.4 μg/ml, 0.08 μg/ml, 0.016 μg/ml and 0.0032 μg/ml, respectively; equal amounts of IMAB362-similar-hG1 and 330CHI chimeric antibodies were added to the control wells, respectively, incubated in a 37° C., 5% CO₂ incubator for 1 hour; the effector cells Jurkat-CD16A/NF were added at an effector-to-target ratio of 20:1, 2×10⁵ effector cells per well, centrifuged at 200 g for 2 minutes to make the effector cells fully contacted with the target cells; incubated in a 37° C., 5% CO₂ incubator for 5-6 hours; fluorescent signals of the well plates were detected with Bio-Turbo firefly luciferase assay kit, the test results are shown in Table 16.

TABLE 16
Determination of the ADCC activities of the humanized
antibodies by luciferase labeling

| Group | EC50 (nM) | Top |
|---|---|---|
| IMAB362-similar-hG1 | 0.69 | 63575 |
| 330CHI | 0.18 | 64473 |
| Hu330-1 | 0.12 | 68271 |
| Hu330-2 | 0.11 | 65144 |

Compared with the IMAB362-similar-hG1 positive antibody, the EC50s of the humanized antibodies Hu330-1 and Hu330-2 were reduced by more than 6 times, and the ADCC activities were basically the same as that of the chimeric antibody. It can be seen that while the humanized antibodies have high affinity, they also show higher ADCC biological activities than that of the positive antibody.

### Example 18 RTCA Determination of the In-Vitro ADCC Cytotoxic Activities of the Humanized Antibodies Against Tumor Cells

Human peripheral blood was collected, from which PBMC cells were isolated, an equal volume of PBS containing 2% fetal bovine serum was added to dilute the blood sample; 15 ml of Lymphoprep density gradient medium for centrifugation (StemCell) was added to a 50 ml centrifuge tube, then the diluted blood sample was added, the centrifuge brake was turned off, centrifuged at 1200 g at room temperature for 10 minutes. After centrifugation, a buffy coat can be seen, which contained enriched PBMC cells, the buffy coat was aspirated and transferred to a new centrifuge tube, the red blood cells were lysed according to the manual of the red blood cell lysis solution (Sigma) at room temperature, washed twice using PBS containing 2 mM EDTA and 2% fetal bovine serum, centrifuged at 400 g at room temperature for 10 min, the supernatant was discarded; the cells were resuspended in 1640 complete culture medium containing 5% fetal bovine serum, the PBMC concentration was adjusted to 3×10⁶ cells/ml; Next, human gastric cancer cells NUGC4 were used as the target cells, treated with the digestion solution of human gastric cancer tissue-derived cells, the cells were collected by centrifugation at 1000 rpm for 5 minutes, the supernatant was discarded; the cells were adjusted to 1.5×10⁵ cells/ml, 100 μl of cell suspension was added to each well in E-Plate 96-well plates, incubated at 37° C., 5% CO₂ for more than 24 hours, the antibody diluted with 1640 culture medium was added to make the final concentration of antibody 2 μg/ml, incubated at 37° C., 5% CO₂ for 1 hour; The effector cells PBMC were added according to an effector to target ratio of 20:1, 3×10⁵ effector cells were added to each well, centrifuged at 200 g for 2 minutes to make the effector cells fully contacted with the target cells; incubated in a 37° C., 5% CO₂ incubator for 21 hours, RTCA analyzer was used to record the impedance value (Cell Index, CI) of the tumor cells in the 96-well plate in real time, the tumor cell cytotoxic percentage of the humanized antibody was calculated through the formula: cell death %=[(control group CI-experimental group CI)/control group CI]×100%.

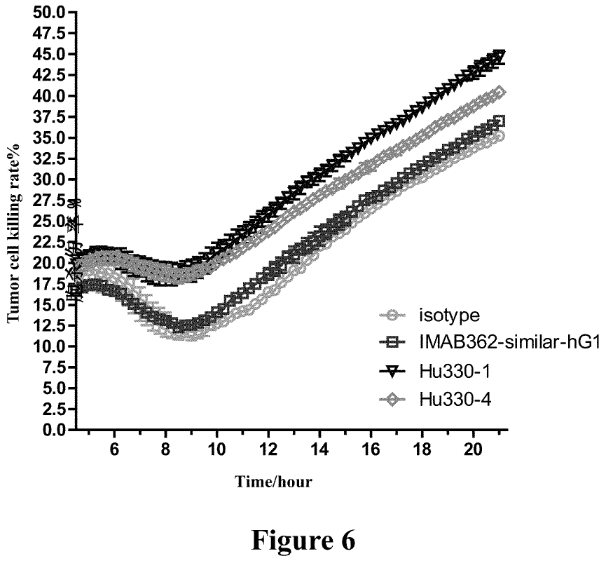

The results are shown in FIG. 6. The tumor cell killing rate of each group increases across time during the 8 to 21 hours of incubation for RTCA real-time detection of the effector cells and target cells. At the 21st hour of in-vitro killing, the humanized antibody Hu330-1 has a maximum cytotoxic rate of 45% against NUGC4 cells, which is 10% higher than the ADCC cytotoxic activity of IMAB362-similar-hG1 positive antibody against tumor cells (P<0.05).

In summary, the humanized antibody Hu330-1 can specifically target and bind to CLDN18.2, and its binding activity and affinity are 15-18 times higher than the positive antibody IMAB362-similar-hG1 at both the molecular level and the cellular level, while the antibody-mediated in-vitro ADCC cytotoxic effect against the target cells shows an antibody concentration-dependency, at lower concentrations, Hu330-1 can already exert a certain cytotoxic effect against tumor cells.

### Example 19 Optimization of the Constant Region of the Humanized Antibody

The variable region sequence of the Hu330-1 humanized antibody was combined with the IgG1 constant region sequence comprising 5 amino acid site mutations, to construct and express the Fc mutant of the humanized antibody Hu330-1-5M (see Table 14 for the sequence); The positive antibody 5M comprising that Fc mutation (see Table 17 for the sequence) was constructed as a control for the Fc fragment.

TABLE 17
Sequence list of the Fc mutant of the humanized antibody

| | Antibody | Amino acid sequence | | Nucleotide sequence | |
|---|---|---|---|---|---|
| No. | name | Heavy chain | Light chain | Heavy chain | Light chain |
| 1 | Hu330-1 | SEQ ID NO | SEQ ID NO | SEQ ID NO | SEQ ID NO |
| | | 92 | 94 | 93 | 95 |
| 2 | Hu330- | SEQ ID NO | SEQ ID NO | SEQ ID NO | SEQ ID NO |
| | 1-5M | 96 | 98 | 97 | 99 |
| 3 | 5M | SEQ ID NO | SEQ ID NO | | |
| | | 100 | 11 | | |

The effect of the Fc-mutated humanized antibody on the binding to the target CLDN18.2 was identified by FACS. NUGC4 cells were digested using the gastric cancer cell digestion solution at 37° C. for 15 minutes; terminated by the complete culture medium, centrifuged at 1000 rpm for 5 minutes, the supernatant was discarded; the cells were resuspended with 2% fetal bovine serum-PBS diluent, the cells were adjusted to 1×10⁶ cells/ml, 100 μl of cell suspension was added to each well of the 96-well U-shaped-bottom plate; the antibody was diluted with 2% fetal bovine serum-PBS to final concentrations of 5 μg/ml, 1 μg/ml, 0.2 μg/ml, 0.04 μg/ml and 0.008 μg/ml, mixed with the cells in equal volume, incubated at 4° C. for 1 hour; centrifuged at 1000 rpm for 5 minutes, the supernatant was discarded; 200 μl of Cell Stain Buffer (Biolegend) was added to each well to wash twice, centrifuged at 1000 rpm for 5 minutes at each time and the supernatant was discarded; 100 μl of PE-anti human Fc antibody (Biolegend) was added, incubated at 4° C. for 1 hour; washed twice using Cell Stain Buffer (Biolegend), centrifuged at 1000 rpm for 5 minutes at each time and the supernatant was discarded. 300 μl of Cell Stain Buffer was added to resuspend the cells, detected with BD FACS Jazz flow analyzer. The results are shown in Table 18.

TABLE 18
FACS detection of the binding activity of the Fc-optimized
humanized antibody

| Group | EC50 (nM) | Top (%) |
|---|---|---|
| IMAB362-similar-hG1 | 9.78 | 58.8 |
| Hu330-1 | 0.80 | 92.1 |
| Hu330-1-5M | 0.76 | 92.4 |

The positive rate of the binding of the Fc-mutated humanized antibody to NUGC4 target cells gradually reaches a platform as the antibody concentration increases, and its binding curve almost completely overlaps with that of the humanized antibody, and the binding activity of the Hu330-1-5M antibody increases by 15 times compared with that of the positive control antibody IMAB362-similar-hG1, which indicates that the amino acid mutation in the Fc fragment of the constant region does not change the specificity and binding activity of the antibody against the CLDN18.2 target.

### Example 20 Detection of the In-Vitro ADCC Cytotoxic Activity of the Optimized Humanized Antibody

1. Determination of the In-Vitro ADCC Activity of the Fc-Mutated Humanized Antibody by Luciferase Labeling

CHO-K1-CLDN18.2 cells (prepared in Example 5) were used as target cells, and the cell density was adjusted to 1×10⁵ cells/ml, inoculated 100 μl/well into 96-well plates, incubated overnight in a 37° C., 5% CO₂ incubator; the antibody was diluted in a 5-fold gradient, making the final antibody concentrations 10 μg/ml, 2 μg/ml, 0.4 μg/ml, 0.08 μg/ml, 0.016 μg/ml and 0.0032 μg/ml, respectively; incubated at 37° C., 5% CO₂ for 1 hour; the effector cells Jurkat-CD16A/NF were added at an effector-to-target ratio of 20:1, 2×10⁵ effector cells per well were added into each well plate, centrifuged at 200 g for 2 minutes to make the effector cells fully contacted with the target cells; incubated in a 37° C., 5% CO₂ incubator for 5-6 hours; fluorescent signals of the well plates were detected with Bio-Turbo firefly luciferase assay kit, the results are shown in Table 19.

TABLE 19
Determination of the ADCC activity of the Fc-optimized
humanized antibody by luciferase labeling

| Group | EC50 (nM) | Top |
|---|---|---|
| IMAB362-similar-hG1 | 1.49 | 89555 |
| Hu330-1 | 0.45 | 89817 |
| Hu330-1-5M | 0.17 | 100434 |
| 5M | 0.44 | 98368 |

Compared with IMAB362-similar-hG1, the Fc-mutated positive antibody (5M) and the humanized Hu330-1 antibody both have an ADCC activity increased by 3 times, while the EC50 of Fc-mutated Hu330-1-5M antibody is 0.17 nM, which is higher than the ADCC activity of the positive control antibody IMAB362-similar-hG1 by more than 8 times, indicating obviously optimized in-vitro ADCC enhancement effect.

2. Determination of the In-Vitro ADCC Cytotoxic Activity of the Fc-Mutated Humanized Antibody Against Tumor Cells by LDH Method

Human peripheral blood was collected, from which PBMC cells were isolated as the effector cells, an equal volume of PBS containing 2% fetal bovine serum was added to dilute the blood sample; 15 ml of Lymphoprep density gradient medium for centrifugation (StemCell) was added to a 50 ml centrifuge tube, then the diluted blood sample was added, the centrifuge brake was turned off, centrifuged at 1200 g at room temperature for 10 minutes. After centrifugation, a buffy coat can be seen, which contained enriched PBMC cells, the buffy coat was aspirated and transferred to a new centrifuge tube. The red blood cells were lysed according to the manual of the red blood cell lysis solution (Sigma) at room temperature, washed twice using PBS containing 2 mM EDTA and 2% fetal bovine serum, centrifuged at 400 g at room temperature for 10 min, the supernatant was discarded; the cells were resuspended using 1640 containing 2% fetal bovine serum prepared using heat-inactivated serum (hereinafter referred to as heat-inactivated culture medium), the PBMC concentration was adjusted to 3×10⁶ cells/ml; human gastric cancer cells NUGC4 were used as the target cells, and treated with the digestion solution of human gastric cancer tissue-derived cells, the cells were collected by centrifugation at 1000 rpm for 5 minutes, the supernatant was discarded; the cells were adjusted to 1.5×10⁵ cells/ml, 100 μl of cell suspension was added to each well of the 96-well plate, incubated at 37° C., 5% CO₂ for 24 hours, the culture medium was aspirated, the antibody diluted with heat-inactivated culture medium was added, making the final concentrations of the antibody 10 μg/ml, 2 μg/ml, 0.4 μg/ml, 0.08 μg/ml, 0.016 μg/ml and 0.0032 μg/ml, incubated at 37° C., 5% CO₂ for 1 hour; the effector cells PBMC were added according to an effector-to-target ratio of 20:1, 3×10⁵ effector cells were added to each well, centrifuged at 200 g for 2 minutes to make the effector cells fully contacted with the target cells; incubated for 22 hours in a 37° C., 5% CO₂ incubator for killing. At the end of the incubation, WST substrate reagent was added to the well plate according to the steps in the manual of the LDH kit (Dojindo Laboratories (Shanghai) Co., Ltd.), color developed under protection from light for 20-30 minutes, the OD450 nm values were determined, and the original data was put into the formula: cell death %=(absorption value in experimental group−spontaneous value of the target cells−value of the effector cells)/(maximum value of the target cells−spontaneous value of the target cells)×100%, absorption value in experimental group and all control groups=absorption value in that group−background value. The results are shown in Table 20.

TABLE 20
Determination of the ADCC activity of the Fc-optimized
humanized antibody by LDH method

| Group | EC50 (nM) | Max (%) |
|---|---|---|
| Isotype | N.A | 52 |
| IMAB362-similar-hG1 | 2.47 | 80.13 |
| Hu330-1-5M | 0.31 | 129.9 |

The Fc-mutated humanized antibody Hu330-1-5M can kill tumor cells NUGC4 in vitro, and its ADCC cytotoxic effect is dose-dependent on the antibody concentration, the calculated EC50 (0.3 nM) is higher than that of IMAB362-similar-hG1 positive antibody by 8 times; at the same time, Hu330-1-5M has a maximum cytotoxic rate of 130% against tumor cells, which exhibits significantly enhanced in-vitro ADCC cytotoxic effect compared to the positive antibody which exhibits 80%.

In summary, the Fc-mutated Hu330-1-5M humanized antibody, on one hand, has an affinity increased by 15 times compared with the positive antibody IMAB362-similar-hG1 in the targeted binding ability to CLDN18.2; and on the other hand, the ability of in-vitro ADCC cytotoxicity against tumor cells is further enhanced after Fc optimization, showing a EC50 activity increased by 8 times than that of the positive antibody, while the maximum cytotoxic effect is increased by more than 60%.

In summary, the CLDN18.2 antibody provided by the present invention has significant binding activity and affinity to CLDN18.2, at the same time it has efficient in-vitro ADCC cytotoxic activity against tumor cells, indicating that the antibody has efficient anti-tumor effects, which can be applied in the preparation of an anti-tumor drug and has broad market prospects.

Although the present invention has been described in detail above, those skilled in the art should understand that various modifications and changes can be made to the present invention without departing from the spirit and scope of the present invention. The scope of the claims of the present invention is not limited to the detailed description made above, but should belong to the claims.

1. (canceled)

2. An anti-Claudin18.2 antibody or the antigen binding fragment thereof, which comprises a heavy chain variable region and a light chain variable region, wherein:
(1) the heavy chain variable region comprises HCDR1 of the amino acid sequence shown in SEQ ID NO 101, HCDR2 of the amino acid sequence shown in SEQ ID NO 105 and HCDR3 of the amino acid sequence shown in SEQ ID NO 106; and
(2) the light chain variable region comprises LCDR1 of the amino acid sequence shown in SEQ ID NO 108, LCDR2 of the amino acid sequence shown in SEQ ID NO 111 and LCDR3 of the amino acid sequence shown in SEQ ID NO 115.

3. The anti-Claudin18.2 antibody or the antigen binding fragment thereof of claim 2, wherein the antibody is a murine-derived antibody, a chimeric antibody or a humanized antibody.

4. The anti-Claudin18.2 antibody or the antigen binding fragment thereof of claim 2, wherein:
(1) the amino acid sequence of the heavy chain variable region is selected from:
an amino acid sequence shown in SEQ ID NO 79, SEQ ID NO 82, SEQ ID NO 88;
and
(2) the amino acid sequence of the light chain variable region is selected from:
an amino acid sequence shown in SEQ ID NO 81, SEQ ID NO 84, SEQ ID NO 86, SEQ ID NO 90.

5. The anti-Claudin18.2 antibody or the antigen binding fragment thereof of claim 4, wherein;
the amino acid sequence of the heavy chain variable region is SEQ ID NO 79, and the amino acid sequence of the light chain variable region is SEQ ID NO 81;
the amino acid sequence of the heavy chain variable region is SEQ ID NO 82, and the amino acid sequence of the light chain variable region is SEQ ID NO 84;
the amino acid sequence of the heavy chain variable region is SEQ ID NO 82, and the amino acid sequence of the light chain variable region is SEQ ID NO 86;
the amino acid sequence of the heavy chain variable region is SEQ ID NO 88, and the amino acid sequence of the light chain variable region is SEQ ID NO 84;
the amino acid sequence of the heavy chain variable region is SEQ ID NO 88, and the amino acid sequence of the light chain variable region is SEQ ID NO 86;
the amino acid sequence of the heavy chain variable region is SEQ ID NO 82, and the amino acid sequence of the light chain variable region is SEQ ID NO 90; or
the amino acid sequence of the heavy chain variable region is SEQ ID NO 88, and the amino acid sequence of the light chain variable region is SEQ ID NO 90.

6. (canceled)

7. (canceled)

8. The anti-Claudin18.2 antibody or the antigen binding fragment thereof of claim 5, wherein:
the amino acid sequence of the heavy chain is SEQ ID NO 92, and the amino acid sequence of the light chain is SEQ ID NO 94; or
the amino acid sequence of the heavy chain is SEQ ID NO 96, and the amino acid sequence of the light chain is SEQ ID NO 98.

9. An isolated polynucleotide, which encodes the anti-Claudin18.2 antibody or the antigen binding fragment thereof of claim 2.

10. The polynucleotide of claim 9, wherein:
(1) the polynucleotide sequence encoding the amino acid sequence of the heavy chain is shown in SEQ ID NO 93 or SEQ ID NO 97; and
(2) the polynucleotide sequence encoding the amino acid sequence of the light chain is shown in SEQ ID NO 95 or SEQ ID NO 99.

11. (canceled)

12. (canceled)

13. (canceled)

14. A pharmaceutical composition, which comprises the anti-Claudin18.2 antibody or the antigen binding fragment thereof of claim 2 and a pharmaceutically acceptable carrier.

15. (canceled)

16. A method of treating a disease comprising administering the anti-Claudin18.2 antibody or the antigen binding fragment thereof of claim 2 to a subject in need there, wherein the disease is selected from gastric cancer, esophageal cancer, pancreatic cancer, lung cancer, ovarian cancer, colon cancer, liver cancer, head and neck cancer, and gallbladder cancer.


SEQUENCE LISTING (116 sequences)

SEQ ID NO: 1 - DNA, 309 bp, Artificial sequence
  Synthetic
gctagcatggacgccatgaagaggggactgtgctgcgtgctgctgctgtgcggagccgtgttcgtgtctcccagcatggaccagtggagcacccaggatctgtacaacaatcctgtgacagccgtgtttaactatcagggcctgtggcggtcctgcgtgagagagagctccggcttcaccgagtgtaggggctactttacactgctgggactgccagcaatgctgcaggccgtgcgcgagcagaagctgatctccgaggaggacctgaattctgccgtggatcatcaccaccaccaccactgactcgag

SEQ ID NO: 2 - protein, 98 aa, Artificial sequence
  Synthetic
MDAMKRGLCCVLLLCGAVFVSPSMDQWSTQDLYNNPVTAVFNYQGLWRSCVRESSGFTECRGYFTLLGLPAMLQAVREQKLISEEDLNSAVDHHHHHH

SEQ ID NO: 3 - DNA, 444 bp, Artificial sequence
  Synthetic
gctagcatggacgccatgaagaggggactgtgctgcgtgctgctgctgtgcggagccgtgttcgtgtctccaagcatggcagtgaccgcatgtcagggactgggctttgtggtgagcctgatcggaatcgcaggaatcatcgcagcaacctgcatggaccagtggtccacacaggatctgtacaacaatcccgtgacagccgtgttcaactatcagggcctgtggcggtcttgcgtgagagagagctccggcttcaccgagtgtaggggctactttacactgctgggactgcctgcaatgctgcaggccgtgcgcgccctgatgatcgtgggaatcgtgctgggagcaatcggactgctggtgtccatctttgagcagaagctgatctccgaggaggacctgaattctgccgtggatcatcaccaccaccaccactgactcgag

SEQ ID NO: 4 - protein, 143 aa, Artificial sequence
  Synthetic
MDAMKRGLCCVLLLCGAVFVSPSMAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFEQKLISEEDLNSAVDHHHHHH

SEQ ID NO: 5 - DNA, 43 bp, Artificial sequence
  Synthetic
agatctatcgactctcgagcgttcttcgttcgttctcacgcgt

SEQ ID NO: 6 - DNA, 798 bp, Artificial sequence
  Synthetic
ggatccatggcagtgacagcatgccagggactgggattcgtggtgtctctgatcggcatcgccggcatcatcgccgccacatgtatggaccagtggagcacccaggatctgtacaacaatccagtgaccgccgtgtttaactatcagggcctgtggcggtcctgcgtgagagagagctccggcttcacagagtgtaggggctactttaccctgctgggactgccagcaatgctgcaggccgtgcgcgccctgatgatcgtgggaatcgtgctgggagcaatcggactgctggtgtctatcttcgccctgaagtgcatcaggatcggcagcatggaggactccgccaaggccaacatgaccctgacatccggcatcatgtttatcgtgtctggactgtgcgcaatcgcaggcgtgagcgtgttcgccaacatgctggtgacaaatttttggatgtccacagccaatatgtacaccggaatgggaggaatggtgcagaccgtgcagacacggtataccttcggcgccgccctgtttgtgggatgggtggcaggaggactgacactgatcggcggcgtgatgatgtgcatcgcatgtagaggactggcaccagaggagaccaattacaaggccgtgagctatcacgcctctggacacagcgtggcatacaagcctggaggcttcaaggcctctacaggctttggcagcaacaccaagaataagaagatctatgacggcggcgccagaaccgaggatgaggtgcagtcctacccctctaagcacgattacgtgtgagaattc

SEQ ID NO: 7 - DNA, 794 bp, Artificial sequence
  Synthetic
ctcgagatggccgtgactgcctgtcagggcttggggttcgtggtttcactgattgggattgcgggcatcattgctgccacctgcatggaccagtggagcacccaagacttgtacaacaaccccgtaacagctgttttcaactaccaggggctgtggcgctcctgtgtccgagagagctctggcttcaccgagtgccggggctacttcaccctgctggggctgccagccatgctgcaggcagtgcgagccctgatgatcgtaggcatcgtcctgggtgccattggcctcctggtatccatctttgccctgaaatgcatccgcattggcagcatggaggactctgccaaagccaacatgacactgacctccgggatcatgttcattgtctcaggtctttgtgcaattgctggagtgtctgtgtttgccaacatgctggtgactaacttctggatgtccacagctaacatgtacaccggcatgggtgggatggtgcagactgttcagaccaggtacacatttggtgcggctctgttcgtgggctgggtcgctggaggcctcacactaattgggggtgtgatgatgtgcatcgcctgccggggcctggcaccagaagaaaccaactacaaagccgtttcttatcatgcctcaggccacagtgttgcctacaagcctggaggcttcaaggccagcactggctttgggtccaacaccaaaaacaagaagatatacgatggaggtgcccgcacagaggacgaggtacaatcttatccttccaagcacgactatgtggatcc

SEQ ID NO: 8 - protein, 271 aa, Artificial sequence
  Synthetic
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGEQKLISEEDLGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 9 - DNA, 794 bp, Artificial sequence
  Synthetic
ctcgagatgtccaccaccacatgccaagtggtggcgttcctcctgtccatcctggggctggccggctgcatcgcggccaccgggatggacatgtggagcacccaggacctgtacgacaaccccgtcacctccgtgttccagtacgaagggctctggaggagctgcgtgaggcagagttcaggcttcaccgaatgcaggccctatttcaccatcctgggacttccagccatgctgcaggcagtgcgagccctgatgatcgtaggcatcgtcctgggtgccattggcctcctggtatccatctttgccctgaaatgcatccgcattggcagcatggaggactctgccaaagccaacatgacactgacctccgggatcatgttcattgtctcaggtctttgtgcaattgctggagtgtctgtgtttgccaacatgctggtgactaacttctggatgtccacagctaacatgtacaccggcatgggtgggatggtgcagactgttcagaccaggtacacatttggtgcggctctgttcgtgggctgggtcgctggaggcctcacactaattgggggtgtgatgatgtgcatcgcctgccggggcctggcaccagaagaaaccaactacaaagccgtttcttatcatgcctcaggccacagtgttgcctacaagcctggaggcttcaaggccagcactggctttgggtccaacaccaaaaacaagaagatatacgatggaggtgcccgcacagaggacgaggtacaatcttatccttccaagcacgactatgtggatcc

SEQ ID NO: 10 - protein, 262 aa, Artificial sequence
  Synthetic
MSTTTCQVVAFLLSILGLAGCIAATGMDMWSTQDLYDNPVTSVFQYEGLWRSCVRQSSGFTECRPYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYVD

SEQ ID NO: 11 - protein, 240 aa, Artificial sequence
  Synthetic
MESQTQVLMSLLFWVSGTCGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 467 aa, Artificial sequence
  Synthetic
MGWSCIILFLVATATGVHSQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 239 aa, Artificial sequence
  Synthetic
METDTLLLWVLLLWVPGSTGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 14 - protein, 466 aa, Artificial sequence
  Synthetic
MGWSWIFLFLLSGTAGVLSQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 15 - DNA, 31 bp, Artificial sequence
  Synthetic
cgcggatccatgtccaccaccacatgccaag

SEQ ID NO: 16 - DNA, 33 bp, Artificial sequence
  Synthetic
ccggaattcttacacatagtcgtgcttggaagg

SEQ ID NO: 17 - DNA, 798 bp, Artificial sequence
  Synthetic
ggatccatgtccaccaccacatgccaagtggtggcgttcctcctgtccatcctggggctggccggctgcatcgcggccaccgggatggacatgtggagcacccaggacctgtacgacaaccccgtcacctccgtgttccagtacgaagggctctggaggagctgcgtgaggcagagttcaggcttcaccgaatgcaggccctatttcaccatcctgggacttccagccatgctgcaggcagtgcgagccctgatgatcgtaggcatcgtcctgggtgccattggcctcctggtatccatctttgccctgaaatgcatccgcattggcagcatggaggactctgccaaagccaacatgacactgacctccgggatcatgttcattgtctcaggtctttgtgcaattgctggagtgtctgtgtttgccaacatgctggtgactaacttctggatgtccacagctaacatgtacaccggcatgggtgggatggtgcagactgttcagaccaggtacacatttggtgcggctctgttcgtgggctgggtcgctggaggcctcacactaattgggggtgtgatgatgtgcatcgcctgccggggcctggcaccagaagaaaccaactacaaagccgtttcttatcatgcctcaggccacagtgttgcctacaagcctggaggcttcaaggccagcactggctttgggtccaacaccaaaaacaagaagatatacgatggaggtgcccgcacagaggacgaggtacaatcttatccttccaagcacgactatgtgtaagaattc

SEQ ID NO: 18 - DNA, 25 bp, Artificial sequence
  Synthetic
atgrasttskggytmarctkgrttt

SEQ ID NO: 19 - DNA, 26 bp, Artificial sequence
  Synthetic
atgraatgsasctgggtywtyctctt

SEQ ID NO: 20 - DNA, 29 bp, Artificial sequence
  Synthetic
atggactccaggctcaatttagttttcct

SEQ ID NO: 21 - DNA, 26 bp, Artificial sequence
  Synthetic
atggctgtcytrgbgctgytcytctg

SEQ ID NO: 22 - DNA, 29 bp, Artificial sequence
  Synthetic
atggvttggstgtggamcttgcyattcct

SEQ ID NO: 23 - DNA, 26 bp, Artificial sequence
  Synthetic
atgaaatgcagctggrtyatsttctt

SEQ ID NO: 24 - DNA, 26 bp, Artificial sequence
  Synthetic
atggrcagrcttacwtyytcattcct

SEQ ID NO: 25 - DNA, 26 bp, Artificial sequence
  Synthetic
atgatggtgttaagtcttctgtacct

SEQ ID NO: 26 - DNA, 20 bp, Artificial sequence
  Synthetic
tgtcaggaactgcaggtgtc

SEQ ID NO: 27 - DNA, 20 bp, Artificial sequence
  Synthetic
ggacagggmtccakagttcc

SEQ ID NO: 28 - DNA, 21 bp, Artificial sequence
  Synthetic
cctggatgacttcagtgttgt

SEQ ID NO: 29 - DNA, 24 bp, Artificial sequence
  Synthetic
atgragwcacakwcycaggtcttt

SEQ ID NO: 30 - DNA, 31 bp, Artificial sequence
  Synthetic
atgaggrcccctgctcagwttyttggwtctt

SEQ ID NO: 31 - DNA, 31 bp, Artificial sequence
  Synthetic
atgggcwtcaagatgragtcacakwyycwgg

SEQ ID NO: 32 - DNA, 29 bp, Artificial sequence
  Synthetic
atgagtgtgcycactcaggtcctggsgtt

SEQ ID NO: 33 - DNA, 31 bp, Artificial sequence
  Synthetic
atgtggggaycgktttyammcttttcaattg

SEQ ID NO: 34 - DNA, 28 bp, Artificial sequence
  Synthetic
atggaagccccagctcagcttctcttcc

SEQ ID NO: 35 - DNA, 28 bp, Artificial sequence
  Synthetic
gtcgttcactgccatcaatcttccactt

SEQ ID NO: 36 - DNA, 357 bp, Artificial sequence
  Synthetic
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgctgatgacttcaagggacggtttgccttctctttggaaacctctgccagcactgcctatttgcagatcaacaacctcaaaaatgaggacatggctacatatttctgtgcaagagacggacagctcgggctacggtttgcttactggggccaagggactctggtcactgtctctgca

SEQ ID NO: 37 - DNA, 336 bp, Artificial sequence
  Synthetic
aacattatgatgacacagtcgccatcatctctggctgtgtctgcaggagaaaaggtcactatgagctgtaagtccagtcaaagtgttttatacagttcaaatcagaagaactacttggcctggtaccagcagaaaccagggcagtctcctaaactgctgatctactgggcatccactagggaatctggtgtccctgatcgcttcacaggcagtggatctgggacagattttactcttaccatcagcagtgtacaagctgaagacctggcagtttattactgtcatcaatacctctcctcgcggacgttcggtggaggcaccaagctggaaatcaaa

SEQ ID NO: 38 - DNA, 355 bp, Artificial sequence
  Synthetic
gaggtgaagctggtggagtctgggggaggcttagtgcagcctggagggtccctgaaactctcctgtgcagcctctggattcactttcagtgactacggaatggcgtgggttcgacaggctccaaggaaggggcctgagtgggtagcattcattagtaatttggcatatagtatctactatgcagacactgtgacgggccgattcaccatctctagagagaatgccaagaacaccctgtacctggaaatgagcagtctgaggtctgaggacacggccatgtattactgtgcaagacttgggtatggtaactcctttgcttactggggccaagggactctggtcactgtctctgcag

SEQ ID NO: 39 - DNA, 340 bp, Artificial sequence
  Synthetic
gacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtgctgggaccaagctggagctgaaac

SEQ ID NO: 40 - DNA, 345 bp, Artificial sequence
  Synthetic
aggttcagctgcagcagtctggagctgagctggcgaggcctggggcttcagtgaagctgtcctgcaaggcttctggctacaccttcacaagctatggtataagctgggtgaagcagagaactggacagggccttgagtggattggagagatttatcctagaagtggtaatacttactacaatgagaagttcaagggcaaggccacactgactgcagacaaatcctccagcacagcgtacatggagctccgcagcctgacatctgaggactctgcggtctatttctgtgcaagccttgcctggtttgcttactggggccaagggactctggtcactgtctctgcag

SEQ ID NO: 41 - DNA, 337 bp, Artificial sequence
  Synthetic
gatattgtgatgactcaggctgcaccctctgtacctgtcactcctggagagtcagtatccatctcctgcaggtctagtaagagtctcctgcatagtaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgtacacgttcggaggggggaccaagctggaaataaaac

SEQ ID NO: 42 - DNA, 355 bp, Artificial sequence
  Synthetic
gaggtccagctgcaacagtctggacctgagctggtgaagcctggggcttcagtgaagatgtcctgcaaggcttctggatacacattcactgactacaacatgcactgggtgaagcagagccatggaaagagccttgagtggattggatatattaaccctaacaatggtggtactagctacaaccagaagttcaagggcaaggccacattgactgtaaacaagtcctccagcacagcctacatggagctccgcagcctgacatcggaggattctgcagtctattactgtgcaaggatctactatggtaactcgtttgcttactggggccaagggactctggtcactgtctctgcag

SEQ ID NO: 43 - DNA, 340 bp, Artificial sequence
  Synthetic
gacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccattcacgttcggctcggggacaaagttggaaataaaac

SEQ ID NO: 44 - DNA, 348 bp, Artificial sequence
  Synthetic
aggtgcagctgaaggagtcaggacctggcctggtggcgccctcacagagcctgtccatcacatgcactgtctcagggttctcattaaccagctatggtgtaagctgggttcgccagcctccaggaaagggtctggagtggctgggagtaatatggggtgacgggagcacaaattatcattcagctctcatatccagactgagcatcagcaaggataactccaagagccaagttttcttaaaactgaacagtctgcaaactgatgacacagccacgtactactgtgccgggggattacgactctggtttgcttactggggccaagggactctggtcactgtctctgcag

SEQ ID NO: 45 - DNA, 340 bp, Artificial sequence
  Synthetic
gacattgtgatgacacagtctccatcctccctggctatgtcagtaggacagaaggtcactatgagctgcaagtccagtcagagccttttaaatagtagcaatcaaaagaactatttggcctggtaccagcagaaaccaggacagtctcctaaacttctggtatactttgcatccactagggaatctggggtccctgatcgcttcataggcagtggatctgggacagatttcactcttaccatcagcagtgtgcaggctgaagacctggcagattacttctgtcagcaacattatagcactccattcacgttcggctcggggacaaagttggaaataaaac

SEQ ID NO: 46 - DNA, 361 bp, Artificial sequence
  Synthetic
caggttcagctgcagcagtctggggctgagctagtgaagcctggagcctcagtgaagatgtcctgcaaggcttctggctacaccttcactacctatcctatagagtggatgaagcagaatcatggaaagagcctagagtggattggaaattttcatccttacaatgatgatactaagtacaatgaaaagttcaagggcaaggccacattgactgtagaaaaatcctctagcacagtctacttggagctcagccgattaacatctgatgactctgctgtttattactgtgcaaggaggggctacggtagtagctacgctatggactactggggtcaaggaacctcagtcaccgtctcctcag

SEQ ID NO: 47 - DNA, 340 bp, Artificial sequence
  Synthetic
gacattgtgatgacacagtctccatcctccctgagtgtgtcagcaggagagaaggtcactatgagctgcaagtccagtcagagtctgttaaacagtggaaatcaaaagaactacttggcctggtaccagcagaaaccagggcagcctcctaaactgttgatctacggggcatccactagggaatctggggtccctgatcgcttcacaggcagtggatctggaaccgatttcactcttaccatcagcagtgtgcaggctgaagacctggcagtttattactgtcagaatgatcatagttatccgtacacgttcggaggggggaccaagctggaaataaaac

SEQ ID NO: 48 - protein, 119 aa, Artificial sequence
  Synthetic
QIQLVQSGPELKKPGETVKISCKASGYTFTNYGMNWVKQAPGKGLKWMGWINTYTGEPTYADDFKGRFAFSLETSASTAYLQINNLKNEDMATYFCARDGQLGLRFAYWGQGTLVTVSA

SEQ ID NO: 49 - protein, 112 aa, Artificial sequence
  Synthetic
NIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSRTFGGGTKLEIK

SEQ ID NO: 50 - protein, 118 aa, Artificial sequence
  Synthetic
EVKLVESGGGLVQPGGSLKLSCAASGFTFSDYGMAWVRQAPRKGPEWVAFISNLAYSIYYADTVTGRFTISRENAKNTLYLEMSSLRSEDTAMYYCARLGYGNSFAYWGQGTLVTVSA

SEQ ID NO: 51 - protein, 113 aa, Artificial sequence
  Synthetic
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 52 - DNA, 355 bp, Artificial sequence
  Synthetic
gaagtgaaactggtggagagcggcggcggcctcgtgcagcctggaggctccctgaagctgagctgtgccgccagcggctttacctttagcgattacggcatggcctgggtgagacaggcccctagaaagggccccgaatgggtggccttcatctccaacctggcctacagcatctactacgccgacaccgtgaccggaagattcaccatctccagagagaacgccaagaacaccctgtacctggagatgagctccctgaggagcgaggacaccgccatgtactactgcgccaggctgggctacggcaacagcttcgcctactggggacagggcaccctggtgacagtgagcgctg

SEQ ID NO: 53 - DNA, 340 bp, Artificial sequence
  Synthetic
gacatcgtgatgacccagagcccctcctccctgacagtgaccgccggcgagaaggtgaccatgagctgcaagagctcccagagcctgctgaacagcggcaatcagaagaactacctgacctggtaccagcagaagcccggccagcctcccaagctgctgatctactgggccagcaccagagagagcggcgtgcctgacagattcaccggcagcggctccggcaccgactttaccctgaccatctccagcgtgcaggccgaggacctggccgtgtactattgccagaacgactacagctaccccctgaccttcggcgccggaaccaagctggagctgaaga

SEQ ID NO: 54 - protein, 115 aa, Artificial sequence
  Synthetic
QVQLQQSGAELARPGASVKLSCKASGYTFTSYGISWVKQRTGQGLEWIGEIYPRSGNTYYNEKFKGKATLTADKSSSTAYMELRSLTSEDSAVYFCASLAWFAYWGQGTLVTVSA

SEQ ID NO: 55 - protein, 112 aa, Artificial sequence
  Synthetic
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPYTFGGGTKLEIK

SEQ ID NO: 56 - DNA, 346 bp, Artificial sequence
  Synthetic
caggtgcagctgcaacaatccggcgccgagctggccaggcctggagcctccgtgaagctgagctgcaaggcctccggctacaccttcacctcctacggcatcagctgggtgaaacagaggacaggccagggcctggagtggattggcgagatctaccccagatccggaaacacctactacaacgagaagttcaagggcaaggctaccctgaccgctgacaagagcagcagcacagcttacatggagctgagatccctgaccagcgaggatagcgccgtctacttctgcgccagcctcgcttggtttgcttactggggccagggaaccctggtgaccgtgagcgctg

SEQ ID NO: 57 - DNA, 337 bp, Artificial sequence
  Synthetic
gacatcgtgatgacccaggctgccccttccgtgcctgtgacccctggagagagcgtgagcatcagctgcaggagcagcaagtccctgctgcacagcaacggcaacacctacctgtactggtttctgcagagacctggccagagcccccagctgctgatctacaggatgagcaatctggcctccggcgtgcccgacagatttagcggctccggcagcggcaccgcctttaccctgaggatcagcagggtggaggccgaggacgtgggcgtgtactactgtatgcagcacctggagtacccctataccttcggcggcggcaccaagctggagatcaaga

SEQ ID NO: 58 - protein, 118 aa, Artificial sequence
  Synthetic
EVQLQQSGPELVKPGASVKMSCKASGYTFTDYNMHWVKQSHGKSLEWIGYINPNNGGTSYNQKFKGKATLTVNKSSSTAYMELRSLTSEDSAVYYCARIYYGNSFAYWGQGTLVTVSA

SEQ ID NO: 59 - protein, 113 aa, Artificial sequence
  Synthetic
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 60 - DNA, 354 bp, Artificial sequence
  Synthetic
gaggtgcagctgcagcagagcggacccgaactggtgaaacccggcgcctccgtgaagatgagctgcaaggccagcggctacaccttcaccgactacaacatgcactgggtgaagcagagccacggcaagtctttagagtggattggctacatcaaccccaacaacggcggcaccagctacaaccagaaattcaagggcaaggccaccctcaccgtgaacaagagctccagcaccgcctacatggagttacgttctttaacctccgaggacagcgccgtgtactactgcgctcgtatctactacggcaacagcttcgcttactggggccaaggtactttagtgaccgtgagcgcc

SEQ ID NO: 61 - DNA, 340 bp, Artificial sequence
  Synthetic
gatatcgtgatgacccagtctccatccagcctgaccgtgacagccggcgagaaggtgaccatgagctgcaagtcttcccagtctctgctgaactccggcaatcagaagaactacctgacatggtatcagcagaagccaggccagccccctaagctgctgatctactgggctagcaccagggagtctggagtgccagaccggttcacaggcagcggctctggcaccgacttcaccctgacaatcagctctgtgcaggccgaggacctggccgtgtactattgccagaatgattactcttatcccttcacatttggctccggcaccaagctggagatcaaga

SEQ ID NO: 62 - protein, 116 aa, Artificial sequence
  Synthetic
QVQLKESGPGLVAPSQSLSITCTVSGFSLTSYGVSWVRQPPGKGLEWLGVIWGDGSTNYHSALISRLSISKDNSKSQVFLKLNSLQTDDTATYYCAGGLRLWFAYWGQGTLVTVSA

SEQ ID NO: 63 - protein, 113 aa, Artificial sequence
  Synthetic
DIVMTQSPSSLAMSVGQKVTMSCKSSQSLLNSSNQKNYLAWYQQKPGQSPKLLVYFASTRESGVPDRFIGSGSGTDFTLTISSVQAEDLADYFCQQHYSTPFTFGSGTKLEIK

SEQ ID NO: 64 - DNA, 348 bp, Artificial sequence
  Synthetic
caagttcagctgaaggagagcggccccggtctggtggctcctagccagtctttaagcatcacttgtaccgtgagcggcttctctttaaccagctacggcgtgagctgggtgagacagcctcccggtaagggtttagagtggctgggagtgatctggggcgacggcagcaccaactaccacagcgctttaatctctcgtctgtccatcagcaaggacaacagcaagagccaagtttttttaaagctgaactctttacagaccgacgacaccgccacctactactgcgccggaggtttaaggctgtggtttgcctactggggccaaggtactttagtgacagtgagcgcc

SEQ ID NO: 65 - DNA, 339 bp, Artificial sequence
  Synthetic
gacatcgtgatgacccagtcccccagctctttagctatgagcgtgggccagaaggtgaccatgagctgcaagtccagccagtctttactgaacagcagcaaccagaagaactatttagcttggtaccagcagaagcccggccagagccccaagctgctggtgtacttcgccagcactcgtgagagcggcgtgcccgatcgtttcatcggcagcggcagcggcaccgatttcactttaaccatcagcagcgtgcaagctgaggatctggccgactacttctgccagcagcactacagcacccccttcaccttcggcagcggcaccaagctggagatcaag

SEQ ID NO: 66 - protein, 120 aa, Artificial sequence
  Synthetic
QVQLQQSGAELVKPGASVKMSCKASGYTFTTYPIEWMKQNHGKSLEWIGNFHPYNDDTKYNEKFKGKATLTVEKSSSTVYLELSRLTSDDSAVYYCARRGYGSSYAMDYWGQGTSVTVSS

SEQ ID NO: 67 - protein, 113 aa, Artificial sequence
  Synthetic
DIVMTQSPSSLSVSAGEKVTMSCKSSQSLLNSGNQKNYLAWYQQKPGQPPKLLIYGASTRESGVPDRFTGSGSGTDFTLTISSVQAEDLAVYYCQNDHSYPYTFGGGTKLEIK

SEQ ID NO: 68 - DNA, 360 bp, Artificial sequence
  Synthetic
caagttcagctgcagcagagcggagccgaactggtgaagcccggtgccagcgtgaagatgagctgcaaggccagcggctacaccttcaccacctaccccatcgagtggatgaagcagaaccacggcaagtctttagagtggatcggcaacttccacccctacaacgacgacaccaagtacaacgagaagttcaagggcaaggccactttaaccgtggagaagagcagcagcaccgtgtatttagagctgagcagactgaccagcgacgacagcgccgtgtactactgcgctcgtagaggctacggcagcagctacgccatggactactggggccaaggtaccagcgtgaccgtgagcagc

SEQ ID NO: 69 - DNA, 339 bp, Artificial sequence
  Synthetic
gacatcgtgatgacccagagccctagctctttaagcgtgagcgccggcgagaaggtgaccatgagctgcaagagcagccagtctttactgaacagcggcaaccagaagaactatttagcttggtaccaacagaagcccggccagccccccaagctgctgatctacggcgccagcaccagagagagcggagtgcccgataggtttaccggcagcggcagcggcaccgacttcactttaaccatcagcagcgtgcaagctgaggatctggccgtgtactactgccagaacgaccacagctacccctacaccttcggcggcggcaccaagctggagatcaag

SEQ ID NO: 70 - protein, 113 aa, Artificial sequence
  Synthetic
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNNYYYPLTFGAGTKLELK

SEQ ID NO: 71 - protein, 113 aa, Artificial sequence
  Synthetic
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSLPFTFGAGTKLELK

SEQ ID NO: 72 - protein, 118 aa, Artificial sequence
  Synthetic
EVKLVESGGGLVQPGGSLKLSCAASGFTFSRYGMAWVRQAPGKGPEWVAFISNSAYSIYYADTVTGRFTISRENAKNTLYLEMSSLRSEDTAMYYCARLGYGNSFAYWGQGTLVTVSA

SEQ ID NO: 73 - protein, 113 aa, Artificial sequence
  Synthetic
DIVMTQSPSSLTVTAGEKVTMSCKSSQSLLNSGNQKNYLTWYQQKPGQPPKLLIYWGSTRESGVPDRFTGSGSGTDFTLTISSVQAEDLAVYYCQNDYSYPLTFGAGTKLELK

SEQ ID NO: 74 - protein, 118 aa, Artificial sequence
  Synthetic
EVKLVESGGGLVQPGGSLKLSCAASGFGFSDYGMAWVRQAPRKGPEWVAFISNSAYSIYYADTVTGRFTISRENAKNTLYLEMSSLRSEDTAMYYCARLGYGNSFAYWGQGTLVTVSA

SEQ ID NO: 75 - protein, 113 aa, Artificial sequence
  Synthetic
DIVMTQSPSSLTVTAGEKVTMSCKSSQSLLNSGNQKNYLTWYQQKPGQPPKLLIYWASTREPGVPDRFTGSGSGTDFTLTISSVQAEDLAVYYCQNDYSYPLTFGAGTKLELK

SEQ ID NO: 76 - protein, 113 aa, Artificial sequence
  Synthetic
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNELFYPLTFGAGTKLELK

SEQ ID NO: 77 - protein, 118 aa, Artificial sequence
  Synthetic
EVKLVESGGGLVQPGGSLKLSCAASGFTFSDYGMAWVRQAPRKGPEWVAFISNLAYSIYYADTVTGRFTISRENAKNTLYLEMSSLRSEDTAMYYCARLGRGNSFAYWGQGTLVTVSA

SEQ ID NO: 78 - protein, 113 aa, Artificial sequence
  Synthetic
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSFPLTFGAGTKLELK

SEQ ID NO: 79 - protein, 118 aa, Artificial sequence
  Synthetic
EVKLVESGGGLVQPGGSLKLSCAASGFGFSDYGMAWVRQAPRKGPEWVAFISNSAYSIYYADTVTGRFTISRENAKNTLYLEMSSLRSEDTAMYYCARLGRGNSFAYWGQGTLVTVSA

SEQ ID NO: 80 - protein, 113 aa, Artificial sequence
  Synthetic
DIVMTQSPSSLTVTAGEKVTMSCKSSQSLLNSGNQKNYLTWYQQKPGQPPKLLIYWGSTRESGVPDRFTGSGSGTDFTLTISSVQAEDLAVYYCQNNYYYPLTFGAGTKLELK

SEQ ID NO: 81 - protein, 113 aa, Artificial sequence
  Synthetic
DIVMTQSPSSLTVTAGEKVTMSCKSSQSLLNSGNQKNYLTWYQQKPGQPPKLLIYWGSTRESGVPDRFTGSGSGTDFTLTISSVQAEDLAVYYCQNELFYPLTFGAGTKLELK

SEQ ID NO: 82 - protein, 118 aa, Artificial sequence
  Synthetic
EVQLVESGGGLVQPGGSLRLSCAASGFGFSDYGMAWVRQAPGKGLEWVSFISNSAYSIYYADTVTGRFTISRDNAKNSLYLQMNSLRAEDTAVYYCARLGRGNSFAYWGQGTLVTVSS

SEQ ID NO: 83 - DNA, 354 bp, Artificial sequence
  Synthetic
gaggtgcagctggtggagagcggcggcggcctggtgcagcccggcggcagcctgagactgagctgcgccgccagcggcttcggcttcagcgactacggcatggcctgggtgagacaggcccccggcaagggcctggagtgggtgagcttcatcagcaacagcgcctacagcatctactacgccgacaccgtgaccggcagattcaccatcagcagagacaacgccaagaacagcctgtacctgcagatgaacagcctgagagccgaggacaccgccgtgtactactgcgccagactgggcagaggcaacagcttcgcctactggggccagggcaccctggtgaccgtgagcagc

SEQ ID NO: 84 - protein, 113 aa, Artificial sequence
  Synthetic
DIVMTQSPDSLAVSLGERATINCKSSQSLLNSGNQKNYLTWYQQKPGQPPKLLIYWGSTRESGVPDRFSGSGSGTDFTLTISSLQAEDVAVYYCQNELFYPLTFGQGTKLEIK

SEQ ID NO: 85 - DNA, 339 bp, Artificial sequence
  Synthetic
gacatcgtgatgacccagagccccgacagcctggccgtgagcctgggcgagagagccaccatcaactgcaagagcagccagagcctgctgaacagcggcaaccagaagaactacctgacctggtaccagcagaagcccggccagccccccaagctgctgatctactggggcagcaccagagagagcggcgtgcccgacagattcagcggcagcggcagcggcaccgacttcaccctgaccatcagcagcctgcaggccgaggacgtggccgtgtactactgccagaacgagctgttctaccccctgaccttcggccagggcaccaagctggagatcaag

SEQ ID NO: 86 - protein, 113 aa, Artificial sequence
  Synthetic
EIVMTQSPPTLSLSPGERVTLSCKSSQSLLNSGNQKNYLTWYQQKPGQAPRLLIYWGSTRESSIPARFSGSGSGTDFTLTISSLQPEDLAVYYCQNELFYPLTFGQGTKLEIK

SEQ ID NO: 87 - DNA, 339 bp, Artificial sequence
  Synthetic
gagatcgtgatgacccagagcccccccaccctgagcctgagccccggcgagagagtgaccctgagctgcaagagcagccagagcctgctgaacagcggcaaccagaagaactacctgacctggtaccagcagaagcccggccaggcccccagactgctgatctactggggcagcaccagagagagcagcatccccgccagattcagcggcagcggcagcggcaccgacttcaccctgaccatcagcagcctgcagcccgaggacctggccgtgtactactgccagaacgagctgttctaccccctgaccttcggccagggcaccaagctggagatcaag

SEQ ID NO: 88 - protein, 118 aa, Artificial sequence
  Synthetic
EVQLLESGGGLVQPGGSLRLSCAASGFGFSDYGMAWVRQAPGKGLEWVSFISNSAYSIYYADTVTGRFTISRDNSKNTLYLQMNSLRAEDTAVYYCARLGRGNSFAYWGQGTLVTVSS

SEQ ID NO: 89 - DNA, 354 bp, Artificial sequence
  Synthetic
gaggtgcagctgctggagagcggcggcggcctggtgcagcccggcggcagcctgagactgagctgcgccgccagcggcttcggcttcagcgactacggcatggcctgggtgagacaggcccccggcaagggcctggagtgggtgagcttcatcagcaacagcgcctacagcatctactacgccgacaccgtgaccggcagattcaccatcagcagagacaacagcaagaacaccctgtacctgcagatgaacagcctgagagccgaggacaccgccgtgtactactgcgccagactgggcagaggcaacagcttcgcctactggggccagggcaccctggtgaccgtgagcagc

SEQ ID NO: 90 - protein, 113 aa, Artificial sequence
  Synthetic
DIVMTQSPLSLPVTLGQPASISCKSSQSLLNSGNQKNYLTWYQQRPGQSPRLLIYWGSTRESGVPDRFSGSGSGTDFTLKISRVEAEDVGVYYCQNELFYPLTFGQGTKLEIK

SEQ ID NO: 91 - DNA, 339 bp, Artificial sequence
  Synthetic
gacatcgtgatgacccagagccccctgagcctgcccgtgaccctgggccagcccgccagcatcagctgcaagagcagccagagcctgctgaacagcggcaaccagaagaactacctgacctggtaccagcagagacccggccagagccccagactgctgatctactggggcagcaccagagagagcggcgtgcccgacagattcagcggcagcggcagcggcaccgacttcaccctgaagatcagcagagtggaggccgaggacgtgggcgtgtactactgccagaacgagctgttctaccccctgaccttcggccagggcaccaagctggagatcaag

SEQ ID NO: 92 - protein, 468 aa, Artificial sequence
  Synthetic
METDTLLLWVLLLWVPGSTGEVQLVESGGGLVQPGGSLRLSCAASGFGFSDYGMAWVRQAPGKGLEWVSFISNSAYSIYYADTVTGRFTISRDNAKNSLYLQMNSLRAEDTAVYYCARLGRGNSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - DNA, 1422 bp, Artificial sequence
  Synthetic
gaattcgccgccaccatggagaccgacaccctgctgctgtgggtgctgctgctgtgggtgcccggatccaccggcgaggtgcagctggtggagagcggcggcggcctggtgcagcccggcggcagcctgagactgagctgcgccgccagcggcttcggcttcagcgactacggcatggcctgggtgagacaggcccccggcaagggcctggagtgggtgagcttcatcagcaacagcgcctacagcatctactacgccgacaccgtgaccggcagattcaccatcagcagagacaacgccaagaacagcctgtacctgcagatgaacagcctgagagccgaggacaccgccgtgtactactgcgccagactgggcagaggcaacagcttcgcctactggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagaaggttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 94 - protein, 240 aa, Artificial sequence
  Synthetic
METDTLLLWVLLLWVPGSTGDIVMTQSPDSLAVSLGERATINCKSSQSLLNSGNQKNYLTWYQQKPGQPPKLLIYWGSTRESGVPDRFSGSGSGTDFTLTISSLQAEDVAVYYCQNELFYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - DNA, 738 bp, Artificial sequence
  Synthetic
gaattcgccgccaccatggagaccgacaccctgctgctgtgggtgctgctgctgtgggtgcccggatccaccggcgacatcgtgatgacccagagccccgacagcctggccgtgagcctgggcgagagagccaccatcaactgcaagagcagccagagcctgctgaacagcggcaaccagaagaactacctgacctggtaccagcagaagcccggccagccccccaagctgctgatctactggggcagcaccagagagagcggcgtgcccgacagattcagcggcagcggcagcggcaccgacttcaccctgaccatcagcagcctgcaggccgaggacgtggccgtgtactactgccagaacgagctgttctaccccctgaccttcggccagggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 96 - protein, 467 aa, Artificial sequence
  Synthetic
MGWSCIILFLVATATGVHSEVQLVESGGGLVQPGGSLRLSCAASGFGFSDYGMAWVRQAPGKGLEWVSFISNSAYSIYYADTVTGRFTISRDNAKNSLYLQMNSLRAEDTAVYYCARLGRGNSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 97 - DNA, 1422 bp, Artificial sequence
  Synthetic
cctagggccaccatgggatggtcctgcatcattctcttcctggtggccaccgctaccggagtgcacagcgaggtgcagctggtggagagcggcggcggcctggtgcagcccggcggcagcctgagactgagctgcgccgccagcggcttcggcttcagcgactacggcatggcctgggtgagacaggcccccggcaagggcctggagtgggtgagcttcatcagcaacagcgcctacagcatctactacgccgacaccgtgaccggcagattcaccatcagcagagacaacgccaagaacagcctgtacctgcagatgaacagcctgagagccgaggacaccgccgtgtactactgcgccagactgggcagaggcaacagcttcgcctactggggccagggcaccctggtgaccgtgagcagcgcttcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcctgcccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcccgaggagcagtacaacagcacgctgcgtgtggtcagcatcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctctggtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtatac

SEQ ID NO: 98 - protein, 240 aa, Artificial sequence
  Synthetic
MESQTQVLMSLLFWVSGTCGDIVMTQSPDSLAVSLGERATINCKSSQSLLNSGNQKNYLTWYQQKPGQPPKLLIYWGSTRESGVPDRFSGSGSGTDFTLTISSLQAEDVAVYYCQNELFYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - DNA, 743 bp, Artificial sequence
  Synthetic
gatatcgccaccatggagtcccagacacaggtgctgatgagcctgctgttctgggtgtccggcacctgtggcgacatcgtgatgacccagagccccgacagcctggccgtgagcctgggcgagagagccaccatcaactgcaagagcagccagagcctgctgaacagcggcaaccagaagaactacctgacctggtaccagcagaagcccggccagccccccaagctgctgatctactggggcagcaccagagagagcggcgtgcccgacagattcagcggcagcggcagcggcaccgacttcaccctgaccatcagcagcctgcaggccgaggacgtggccgtgtactactgccagaacgagctgttctaccccctgaccttcggccagggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgattaattaa

SEQ ID NO: 100 - protein, 467 aa, Artificial sequence
  Synthetic
MGWSCIILFLVATATGVHSQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSILTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 101 - protein, 8 aa, Artificial sequence
  Synthetic
GFGFSDYG

SEQ ID NO: 102 - protein, 8 aa, Artificial sequence
  Synthetic
GFTFSDYG

SEQ ID NO: 103 - protein, 8 aa, Artificial sequence
  Synthetic
GFTFSRYG

SEQ ID NO: 104 - protein, 8 aa, Artificial sequence
  Synthetic
ISNLAYSI

SEQ ID NO: 105 - protein, 8 aa, Artificial sequence
  Synthetic
ISNSAYSI

SEQ ID NO: 106 - protein, 11 aa, Artificial sequence
  Synthetic
ARLGRGNSFAY

SEQ ID NO: 107 - protein, 11 aa, Artificial sequence
  Synthetic
ARLGYGNSFAY

SEQ ID NO: 108 - protein, 17 aa, Artificial sequence
  Synthetic
KSSQSLLNSGNQKNYLT

SEQ ID NO: 109 - protein, 7 aa, Artificial sequence
  Synthetic
WASTREP

SEQ ID NO: 110 - protein, 7 aa, Artificial sequence
  Synthetic
WASTRES

SEQ ID NO: 111 - protein, 7 aa, Artificial sequence
  Synthetic
WGSTRES

SEQ ID NO: 112 - protein, 9 aa, Artificial sequence
  Synthetic
QNDYSFPLT

SEQ ID NO: 113 - protein, 9 aa, Artificial sequence
  Synthetic
QNDYSLPFT

SEQ ID NO: 114 - protein, 9 aa, Artificial sequence
  Synthetic
QNDYSYPLT

SEQ ID NO: 115 - protein, 9 aa, Artificial sequence
  Synthetic
QNELFYPLT

SEQ ID NO: 116 - protein, 9 aa, Artificial sequence
  Synthetic
QNNYYYPLT